(19) United States
(12) Patent Application Publication
(10) Pub. No.: US 2007/0212705 A1
(43) Pub. Date: Sep. 13, 2007

(54) PSEUDOMONAS AERUGINOSA L-D CARBOXYPEPTIDASE A, EXPRESSION AND ACTIVITY

(76) Inventor: Ellen Z. Baum, Randolph, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/550,868

(22) Filed: Oct. 19, 2006

Related U.S. Application Data
(60) Provisional application No. 60/730,192, filed on Oct. 25, 2005.

(51) Int. Cl. C12Q 1/68; C07H 21/04; C12P 21/06; C12N 9/48; C12N 15/74; C12N 1/21

(52) U.S. Cl. 435/6; 435/69.1; 435/212; 435/252.34; 435/471; 536/23.2

(57) ABSTRACT

The present invention provides nucleic acid and polypeptide sequences of a L, D carboxypeptidase from Pseudomonas aeruginosa (PA LDC). The isolated nucleic acid or polypeptide molecules of the invention can be used in detection assays screening and drug discovery assays.

### CROSS REFERENCE TO RELATED APPLICATIONS

This application claims priority of the benefits of the filing of U.S. Application Ser. No. 60/730,192, filed Oct. 25, 2005. The complete disclosures of the aforementioned related U.S. patent application are hereby incorporated herein by reference for all purposes.

### FIELD OF THE INVENTION

The present invention relates to L-D carboxypeptidase enzymes. In particular, the present invention relates to isolated nucleic acid molecules and polypeptides of a novel P. aeruginosa L-D carboxypeptidase A (PA LDC), and uses thereof.

### BACKGROUND

Bacterial cell wall murein peptide is recycled, in that the degradation products are re-incorporated into new cell walls. It is estimated that as much as 50% of the total cell wall murein peptide is recycled in each bacterial generation (Goodell E W and Schwarz U., Release of cell wall peptides into culture medium by exponentially growing Escherichia coli. Journal of Bacteriology. 162(1):391-7.1985). The enzyme L-D carboxypeptidase A (LDC) cleaves the peptide bond between the dibasic amino acid and the C-terminal D-alanine residue of cell wall tetrapeptides (Templin, M F., Ursinus, A., and Höltje, J V. A defect in cell wall recycling triggers autolysis during the stationary growth phase of Escherichia coli. EMBO Journal 18, 4108-4117. 1999). The dibasic amino acids comprise L-lysine in gram positive bacteria and meso-diaminopimelic acid in gram negative bacteria. The LDC enzyme from Escherichia coli was cloned by Templin et al. (Templin et al. 1999). It was shown that LDC is essential for survival of Escherichia coli as deletion of the gene causes autolysis during the stationary phase of bacterial growth (Templin et al.1999). This finding has particular relevance to antibiotic research, making LDC an appropriate target for the inhibition of bacterial growth and for the treatment bacterial infections.

Proteases are usually classified according to three major criteria. The three major criteria currently used for the classification of peptidases are: (i) the reaction catalyzed, (ii) the chemical nature of the catalytic site, and (iii) the evolutionary relationship, as revealed by the structure. Proteases can be further divided into families and clans after initial classification according to the three criteria listed above. In reference to classification by the reactions they catalyse, proteases may be divided into to sub-subclasses of peptide hydrolases depending on the location of the enzymatic action, either exopeptidase or endopeptidase. Exopeptidases cleave peptide bonds at the amino terminus (aminopeptidase) or the carboxy terminus (carboxypeptidase) of a peptide substrate. Endopeptidases, on the other hand, cleave peptide bonds internally, away from either termini of the protein substrate.

Exopeptidases may be subdivided on the basis of catalytic mechanism. Serine-type peptidases have an active center serine involved in the catalytic process, the cysteine-type peptidases have a cysteine residue in the active center, the aspartic-type endopeptidases depend on two aspartic acid residues for their catalytic activity, and the metallopeptidases use a metal ion (commonly zinc) in the catalytic mechanism. A number of endopeptidases cannot yet be assigned to any of the enzyme sub-subclasses.

LDC or muramoyl-tetrapeptide carboxypeptidase is classified as part of the U61 peptidase family, however their catalytic mechanism is currently unknown and few inhibitors of LDC have been identified. LDC enzymes are weak inhibitors, in that relatively high concentrations (100 μg/ml) are required for inhibition of the enzyme (Templin et al., 1999, supra). LDC is inhibited by Nocardicin A, a β-lactam with a D-amino acid side chain (Templin et al., 1999, supra); this compound binds to LDC and can be used to purify the enzyme by affinity chromatography (Ursinus A, Steinhaus H, Holtje J V. Purification of a nocardicin A-sensitive LD-carboxypeptidase from Escherichia coli by affinity chromatography. Journal of Bacteriology. 174(2):441-6. 1992).

Since the majority of known antibiotics act during cell growth, Templin's work suggested that inhibition of the LDC enzyme during stationary phase might provide a novel, useful means to kill non-growing cells, either alone, or in combination with an antibiotic which works during the growing phase. In particular, the bacterial pathogen Pseudomonas aeruginosa forms biofilms of stationary phase cells that are difficult to treat with conventional antibiotics (Costerton, J. W., Stewart. P. S., and Greenberg, E. P. Bacterial biofilms: a common cause of persistent infections. Science 284, 1318-1322. 1999).

Biofilms are aggregates of bacteria enmeshed in an extracellular polymeric matrix which they synthesize. Biofilms tend to form on solid surfaces and pose problems both in the environment (e.g., corrosion of metal pipes) and in medicine. In the human body, biofilms can form on foreign bodies, including medical devices such as catheters, sutures, contact lenses, orthopedic devices, mechanical heart valves, shunts, grafts, etc., and on dead tissue in the human body (Parsek, M. R., and Fuqua, C. Biofilms 2003: Emerging themes and challenges in studies of surface-associated microbial life. Journal of Bacteriology. 186, 4427-4440. 2004). Biofilms are responsible for a variety of chronic infections, including cystic fibrosis airway infections caused by P. aeruginosa, and other types of infections including some instances of dental caries, periodontitis, otitis media, necrotizing fascitis, musculoskeletal infections, as well as, biliary tract infections (Costerton et al. 1999). It is well known that biofilms of P. aeruginosa are refractory to antibiotic treatment (Costerton et al. 1999, Parsek and Fuqua, 2004). Several features of the biofilms are thought to give rise to this heightened resistance, including decreased penetration of antibiotic through the entire biofilm, slow metabolic activity of cells within the biofilm, and phenotypic variability within the biofilm (Costerton et al. 1999, Parsek and Fuqua, 2004, supra).

There is a need to identify additional antibiotics that act during the stationary phase of bacterial growth, as these antibiotics could treat a range of bacterial infections, including biofilms. Often after most of the bacteria present in the biofilm have been killed by antibiotics, a small population of “persisters” remain which can give rise to new cells, therefore there also exists a need to reduce the viability of these persisters. Additionally, there is a need for systems that can be used to test compounds that potentially inhibit LDC gene expression and LDC enzyme activity. Identification and testing of such compounds would enable the treatment of various infectious diseases such as those which occur in cystic fibrosis patients, medical device-related infections associated with bacterial infection or for uses in other conditions where inhibition or regulation of bacterial growth is desirable. Further there is a need to treat infections of Pseudomonas aeruginosa and other biofilm forming bacteria. Additionally there exists a need to apply an LDC inhibitor to the surface of, or incorporate the inhibitor into the mixture of items such as, medical devices to prevent or treat biofilms and reduce the viability of persisters.

### SUMMARY OF THE INVENTION

In one general aspect the invention provides a substantially purified polypeptide capable of LDC activity and having at least 96% sequence identity to SEQ ID NO:2.

In another general aspect the invention provides a nucleic acid probe of at least 30 nucleotides but less than 924 nucleotides that selectively hybridizes to a polynucleotide encoded by SEQ ID NO: 1 under stringent hybridization conditions.

In a further general aspect the invention provides a nucleic acid probe selected from: SEQ ID NO:3 and SEQ ID NO:4, that selectively hybridizes to a polynucleotide encoded by SEQ ID NO: 1 under stringent hybridization conditions.

In some embodiments the invention provides a polypeptide corresponding to 20 contiguous amino acids of SEQ ID NO:2.

In other embodiments the invention provides an antibody that specifically binds to a polypeptide capable of LDC activity and wherein the polypeptide has at least 96% sequence identity to SEQ ID NO:2.

In some aspects the invention further provides an antibody, wherein the antibody is a polyclonal antibody or a monoclonal antibody.

In other general aspects the invention provides a kit comprising an antibody of a polyclonal antibody or a monoclonal antibody that specifically binds to a polypeptide capable of LDC activity and wherein the polypeptide has at least 96% sequence identity to SEQ ID NO:2.

The invention additionally provides a method for detecting PA LDC polynucleotide in a sample, comprising the steps of: (a) contacting the polynucleotide with a nucleic acid probe of at least 30 nucleotides but less than 924 nucleotides that selectively hybridizes to the polynucleotide under stringent hybridization conditions; and (b) detecting the polynucleotide with the nucleic acid probe.

In other embodiments the invention provides a method of detecting a polypeptide capable of LDC activity and having at least 96% sequence identity to SEQ ID NO: 2. comprising the steps of: (a) contacting the polypeptide with an antibody that specifically binds to a polypeptide capable of LDC activity and having at least 96% sequence identity to SEQ ID NO: 2 and (b) detecting the polypeptide with the antibody.

In other general aspects the invention provides a method of identifying a compound that decreases the activity of an LDC enzyme, comprising the steps of: (a) contacting the LDC enzyme with a test compound; and (b) detecting the level of enzyme activity.

In further aspects the invention provides a method of wherein the LDC enzyme comprises a polypeptide having an amino acid sequence of SEQ ID NO: 2.

The invention additionally provides methods wherein the contacting step further comprises the step of contacting the test compound with a cell expressing the LDC enzyme.

In other aspects the invention further provides a method wherein the cell encodes a recombinant LDC enzyme.

In some embodiments the invention provides a method wherein the cell endogenously expresses an LDC enzyme.

Additionally the invention provides a method of identifying a compound that decreases the activity of an LDC enzyme, comprising the steps of: a) contacting the LDC enzyme with a test compound; b) incubating the LDC enzyme in a buffer solution comprising a substrate; c) adding a second enzyme to produce hydrogen peroxide; d) adding a third enzyme and a fluorogenic horseradish peroxidase substrate to react with the hydrogen peroxide; and e) measuring a resulting product in a fluorometer In other aspects the invention further provides a method comprising incubating the enzyme at a LDC enzyme-activating temperature from about 35° C. to about 39° C.

The invention also provides a method identifying a compound that decreases the activity of an LDC enzyme wherein the second enzyme is D-amino acid oxidase.

The invention further provides a method identifying a compound that decreases the activity of an LDC enzyme, wherein the third enzyme is horse radish peroxidase.

In other aspects the invention provide a method identifying a compound that decreases the activity of an LDC enzyme, wherein the fluorogenic horseradish peroxidase substrate is Amplex Red.

Additionally the invention provides a method identifying a compound that decreases the activity of an LDC enzyme, wherein the LDC enzyme is of Pseudomonas aeruginosa.

In some embodiments the invention provides a method identifying a compound that decreases the activity of an LDC enzyme, wherein the LDC enzyme is of E. coli.

In other embodiments the invention provides a method of identifying a compound useful for causing bacterial lysis in the stationary phase of bacterial growth, comprising the steps of: a) contacting an LDC enzyme with a test compound; b) determining whether the test compound decreases the activity of the enzyme; c) administering the test compound to a bacterial culture; and d) measuring bacterial cell lysis in the culture.

Additionally the invention provides a method of identifying a compound useful for inhibiting bacterial growth or viability, comprising the steps of: a) contacting a PA LDC enzyme with a test compound; b) determining whether the test compound decreases the activity of the enzyme; c) administering the test compound to an animal; and d) determining the extent to which the test compound reduces the bacterial infection of the animal.

In some aspects the invention provides a method of inducing bacterial lysis, comprising one or more of the steps selected from: a) decreasing the expression of a PA LDC gene; and b) contacting a PA LDC enzyme with a compound which decreases the activity of the enzyme.

In other aspects the invention provides a method for inhibiting bacterial growth or viability, comprising one or more of the steps selected from: a) decreasing the expression of a PA LDC gene; and b) contacting a PA LDC enzyme with a compound which decreases the activity of the enzyme.

In some embodiments the invention provides a method for inhibiting bacterial growth or viability, comprising administering an effective dose of a PA LDC inhibitor selected from: a) 2-Benzofurancarbothioic acid, S,S′-2,3-quinoxalinediyl ester, and b) 3,4-dihydro-g,3-dioxo-2H-1,4-Benzoxazine-6-crotonic acid.

In other embodiments the invention provides a method of inducing bacterial lysis, comprising administering an effective dose of a PA LDC inhibitor selected from:

(a) 2-Benzofurancarbothioic acid, S,S′-2,3-quinoxalinediyl ester, and

(b) 3,4-dihydro-g,3-dioxo-2H-1,4-Benzoxazine-6-crotonic acid.

In some aspects the invention provides a method of treating a Pseudomonas aeruginosa infection in a patient, comprising administering an effective dose of a PA LDC inhibitor selected from: a) 2-Benzofurancarbothioic acid, S,S′-2,3-quinoxalinediyl ester, and b) 3,4-dihydro-g,3-dioxo-2H-1,4-Benzoxazine-6-crotonic acid.

In other aspects the invention provides a device comprising a therapeutically effective amount of an LDC inhibitor effective to inhibit bacterial growth wherein the device is selected from: a catheter, a stent, a suture, a contact lens, an orthopedic device, a mechanical heart valve, a shunt, and a graft.

Other aspects, features and advantages of the invention will be apparent from the following disclosure, including the detailed description of the invention and its preferred embodiments and the appended claims.

### BRIEF DESCRIPTION OF THE FIGURES

FIG. 1, illustrates results of an amino acid sequence alignment of the E. coli LDC protein (muramoyl-tetrapeptide carboxypeptidase P76008, classified as a peptidase with an unknown catalytic mechanism) with the protein PA5198 from P. aeruginosa. The consensus amino acids are highlighted and shown below the aligned sequences. The amino acid sequences of these two proteins were then aligned using the Vector NTI program (InforMax, Inc., Invitrogen Corp., Carlsbad, Calif.), which indicated 25% identity and 35% similarity (the latter accepts similar amino acids at a given position) in comparing the two proteins.

FIG. 2, illustrates the P. aeruginosa open reading frame AAG08583.1 from strain PA01 of P. aeruginosa which was amplified by polymerase chain reaction and cloned into the NdeI/BamHI sites of the N-terminal his-tagged protein expression vector pET14b (Novagen, EMD Biosciences, Madison, Wis.). The actual start “ATG” and stop “TAG” codons are shown in bold. DNA sequence analysis (ACGT, Inc., Wheeling, IL) confirmed the complete identity of the cloned gene (now called PA LDC) with the P. aeruginosa strain PA01 sequence in Genbank (Accession number AE004932). FIG. 3, illustrates the cleavage of a tetrapeptide substrate (L-Ala-γ-D-Glu-L-Lys-D-Ala) by E. coli LDC (squares) and P. aeruginosa LDC (circles). The P. aeruginosa LDC enzyme cleaved the substrate, with about two-fold lower specific activity compared to the E. coli enzyme. Relative fluorescence units (RFU) shown on the y-axis are produced from the Amplex Red detection system (Molecular Probes, Eugene, Oreg.) and indicate the relative amounts of D-alanine produced from the cleavage of the polypeptide substrate. The amount of the LDC added is shown in nanograms per well on the x-axis.

FIG. 4, illustrates the inhibition of PA LDC activity (E. coli IC₅₀=1.4 μM) using the compound, 2-Benzofurancarbothioic acid, S,S′-2,3-quinoxalinediyl ester, CAS Registry #253328-58-4. To demonstrate the feasibility of detecting inhibitors of PA LDC using the purified PA LDC enzyme and the aforementioned detection system, several inhibitors previously shown to be inhibitory to E. coli LDC were tested against PA LDC. PA LDC was incubated together with 2-Benzofurancarbothioic acid, S,S′-2,3-quinoxalinediyl ester for 10 minutes at room temperature, prior to addition of a tetrapeptide substrate. RFU were determined with the Amplex Red detection system, and percent inhibition was calculated by setting the RFU of the control samples (i.e., without 2-Benzofurancarbothioic acid, S,S′-2,3-quinoxalinediyl ester to 0% inhibition).

### DETAILED DESCRIPTION

All publications cited herein are hereby incorporated by reference. Unless defined otherwise, all technical and scientific terms used herein have the same meaning as commonly understood to one of ordinary skill in the art to which this invention pertains.

“An activity”, “a biological activity”, or “a functional activity” of a polypeptide or nucleic acid refers to an activity exerted by a polypeptide or nucleic acid molecule as determined in vivo, or in vitro, according to standard techniques. Such activities can be a direct activity, such as an association with or an enzymatic activity on a second protein, an ion enzyme activity, or an indirect activity, such as a cellular signaling activity mediated by interaction of the protein with one or more than one additional protein or other molecule(s), including, but not limited to, interactions that occur in a multi-step, serial fashion.

A “biological sample” as used herein refers to a sample containing or consisting of cell or tissue matter, such as cells or biological fluids isolated from a subject. The “subject” can be a mammal, such as a rat, a mouse, a monkey, a human, or any other organism, that has been the object of treatment, observation or experiment. Examples of biological samples include, for example, sputum, blood, blood cells (e.g., white blood cells), amniotic fluid, plasma, semen, bone marrow, tissue or fine-needle biopsy samples, urine, peritoneal fluid, pleural fluid, and cell cultures. Biological samples can also include sections of tissues such as frozen sections taken for histological purposes. Biological samples can comprise any bacterial species. A test biological sample is the biological sample that has been the object of analysis, monitoring, or observation. A control biological sample can be either a positive or a negative control for the test biological sample. Often, the control biological sample contains the same type of tissues, cells and/or biological fluids of interest as that of the test biological sample.

The term “stationary phase” as used herein refers to a general plateau of the growth curve after log growth in a culture, during which the cell number remains relatively constant and cells are produced at relatively the same rate as older cells die.

A “cell” refers to at least one cell or a plurality of cells appropriate for the sensitivity of the detection method. Cells suitable for the present invention can be bacterial, for example Pseudomonas aeruginosa or other prokaryotes.

A “clone” is a population of cells derived from a single cell or common ancestor by mitosis. A “cell line” is derived from clonal expansion of primary cells, and is capable of stable growth in vitro for many generations.

A “DNA clone” is a section of DNA that has been copied, isolated or removed from a host and inserted into a vector molecule, such as a plasmid or a phage, or a chromosome, and then replicated to form many identical copies.

The term “cDNA” as used herein means a complementary DNA (cDNA) Synthetic DNA reverse transcribed from a specific RNA through the action of the enzyme reverse transcriptase. DNA is synthesized by reverse transcriptase using RNA as a template.

A “gene” is a segment of DNA involved in producing a peptide, polypeptide, or protein, and the mRNA encoding such protein species, including the coding region, non-coding regions preceding (“5′UTR”) and following (“3′UTR”) the coding region. A “gene” can also include intervening non-coding sequences (“introns”) between individual coding segments (“exons”). “Promoter” means a regulatory sequence of DNA that is involved in the binding of RNA polymerase to initiate transcription of a gene. Promoters are often upstream (“5′ to”) the transcription initiation site of the gene. A “regulatory sequence” refers to the portion of a gene that can control the expression of the gene. A “regulatory sequence” can include promoters, enhancers and other expression control elements such as polyadenylation signal sites, ribosome binding sites (for bacterial expression), and/or, operators. An “enhancer” means a regulatory sequence of DNA that can regulate the expression of a gene in a distance- and orientation-independent fashion. Enhancers can be located upstream, downstream, or even within the gene they control. A “coding region” refers to the portion of a gene that encodes amino acids and the start and stop signals for the translation of the corresponding polypeptide via triplet-base codons.

“Nucleic acid sequence” or “nucleotide sequence” refers to the arrangement of either deoxyribonucleotide or ribonucleotide residues in a polymer in either single- or double-stranded form. Nucleic acid sequences can be composed of natural nucleotides of the following bases: thymidine, adenine, cytosine, guanine, and uracil; abbreviated T, A, C, G, and U, respectively, and/or synthetic analogs. Synthetic analogs can include nucleotide and nucleoside analaogs as well as non-nucleotide and non-nucleoside analogs.

The term “oligonucleotide” refers to a single-stranded DNA or RNA sequence of a relatively short length, for example, less than 100 residues long. For many methods, oligonucleotides of about 16-40 nucleotides in length are useful, although longer oligonucleotides of greater than about 40 nucleotides can sometimes be utilized. Some oligonucleotides can be used as “primers” for the synthesis of complimentary nucleic acid strands. For example, DNA primers can hybridize to a complementary nucleic acid sequence to prime the synthesis of a complementary DNA strand in reactions using DNA polymerases. Oligonucleotides are also useful for hybridization in several methods of nucleic acid detection, for example, in Northern blotting or in situ hybridization.

The term “probe” as used herein refers to a nucleic acid that can selectively hybridize under stringent hybridization conditions to a nucleic acid molecule comprising a nucleotide sequence that encodes a polypeptide capable of LDC activity and having at least 96% sequence identity to SEQ ID NO: 2. Additionally, a probe can selectively hybridize under stringent hybridization conditions to a polypeptide capable of LDC activity and having at least 96% sequence identity to SEQ ID NO: 2. For example a suitable probe can comprise SEQ ID NO:3 or SEQ ID NO:4 as described in Example 1. Nucleic acid probes can be of any practical length similar to oligonucleotides in general and can be RNA or DNA as described above. For example probes can be from 10-20, 21-40, 41-60, or greater than 61 nucleotides in length. More particularly probes can be from 34 to 37 nucleotides in length such as SEQ ID NO:4 or SEQ ID NO:3. Such probes are useful for the detection of LDC molecules in heterogeneous mixtures or for the amplification of rare copies, genomic copies and recombinant copies in vectors as in the case of polymerase chain reactions, south-westerns and other common molecular biology techniques. Hybridization conditions are well know in the art, for example but not limited to those as described in Sambrook et al., Molecular Cloning: A Laboratory Manual, Second Edition, Cold Spring Harbor Laboratory, Cold Spring Harbor, N.Y., (1989).

A “polypeptide sequence” or “protein sequence” refers to the arrangement of amino acid residues in a polymer. Polypeptide sequences can be composed of the standard 20 naturally occurring amino acids, in addition to rare amino acids and synthetic amino acid analogs. Shorter polypeptides are generally referred to as peptides.

An “isolated” nucleic acid molecule is one that is substantially separated from other nucleic acid molecules present in the natural source of the nucleic acid. An “isolated” nucleic acid molecule can be, for example, a nucleic acid molecule that is free of at least one of the nucleotide sequences that naturally flank the nucleic acid molecule at its 5′ and 3′ ends in the genomic DNA of the organism from which the nucleic acid is derived. Isolated nucleic acid molecules include, without limitation, separate nucleic acid molecules (e.g., cDNA or genomic DNA fragments produced by PCR or restriction endonuclease treatment) substantially independent of other sequences, as well as nucleic acid molecules that are incorporated into a vector, an autonomously replicating plasmid, a virus (e.g., a retrovirus, adenovirus, or herpes virus), or into the genomic DNA of a prokaryote or eukaryote. In addition, an isolated nucleic acid molecule can include a nucleic acid molecule that is part of a hybrid or fusion nucleic acid molecule. An isolated nucleic acid molecule can be a nucleic acid molecule that is: (i) amplified in vitro by, for example, polymerase chain reaction (PCR); (ii) synthesized by, for example, chemical synthesis; (iii) recombinantly produced by cloning; or (iv) purified, as by cleavage and electrophoretic or chromatographic separation.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals that are involved in the synthesis of the protein. Accordingly such preparations of the protein have less than about 30%, 20%, 10%, 5% (by dry weight) of chemical precursors or compounds other than the polypeptide of interest. Isolated biologically active polypeptide can have several different physical forms. An isolated polypeptide can exist as a full-length nascent or unprocessed polypeptide, or as a partially processed polypeptide or as a combination of processed polypeptides. The full-length nascent polypeptide can be postranslationally modified by specific proteolytic cleavage events that result in the formation of fragments of the full-length nascent polypeptide. The full length protein or fragments of the polypeptide can be chemically modified. A fragment, or physical association of fragments can have the biological activity associated with the full-length polypeptide; however, the degree of biological activity associated with individual fragments can vary. An isolated or substantially purified polypeptide, can be a polypeptide encoded by an isolated nucleic acid sequence, as well as a polypeptide synthesized by, for example, chemical synthetic methods, and a polypeptide separated from biological materials, and then purified, using conventional protein analytical or preparatory procedures, to an extent that permits it to be used according to the methods described herein.

The term “selectively hybridize” as used herein refers to the ability of a probe to bind to a particular or intended nucleic acid or polypeptide target sequence or molecule in a heterogeneous mixture of nucleic acids or polypeptides, for example a probe can selectively hybridize to a nucleotide sequence encoding an LDC protein or a polypeptide capable of LDC activity in a heterogeneous mixture.

“Recombinant” refers to a nucleic acid, a protein encoded by a nucleic acid, a cell, or a viral particle, that has been modified using molecular biology techniques to something other than its natural state. For example, recombinant cells can contain nucleotide sequence that is not found within the native (non-recombinant) form of the cell or can express native genes that are otherwise abnormally expressed, under-expressed, or not expressed at all. Recombinant cells can also contain genes found in the native form of the cell wherein the genes are modified and re-introduced into the cell by artificial means. The term also encompasses cells that contain an endogenous nucleic acid molecule that has been modified without removing the nucleic acid from the cell; such modifications include those obtained, for example, by gene replacement, and site-specific mutation.

A “recombinant host cell” is a cell that has had introduced into it a recombinant DNA sequence. Recombinant DNA sequences can be introduced into host cells using any suitable method including, for example, electroporation, calcium phosphate precipitation, microinjection, transformation, biolistics “gene-gun” and viral infection. Recombinant DNA may or may not be integrated (covalently linked) into chromosomal DNA making up the genome of the cell. For example, the recombinant DNA can be maintained on an episomal element, such as a plasmid. Alternatively, with respect to a stably transformed or transfected cell, the recombinant DNA has become integrated into the chromosome so that it is inherited by daughter cells through chromosome replication. This stability is demonstrated by the ability of the stably transformed or transfected cell to establish cell lines or clones comprised of a population of daughter cells containing the exogenous DNA. Recombinant host cells can be prokaryotic or eukaryotic, including bacteria such as Pseudomonas aeruginosa, E. coli, fungal cells such as yeast, mammalian cells such as cell lines of human, bovine, porcine, monkey and rodent origin, and insect cells such as Drosophila- and silkworm-derived cell lines. It is further understood that the term “recombinant host cell” refers not only to the particular subject cell, but also to the progeny or potential progeny of such a cell. Because certain modifications can occur in succeeding generations due to either mutation or environmental influences, such progeny cannot, in fact, be identical to the parent cell, but are still included within the scope of the term as used herein.

The term “vector” or “construct” refers to a nucleic acid molecule into which a heterologous nucleic acid can be or is inserted. Some vectors can be introduced into a host cell allowing for replication of the vector or for expression of a protein that is encoded by the vector or construct. Vectors typically have selectable markers, for example, genes that encode proteins allowing for drug resistance, origins of replication sequences, and multiple cloning sites that allow for insertion of a heterologous sequence. Vectors are typically plasmid-based and are designated by a lower case “p” followed by a combination of letters and/or numbers. Starting plasmids disclosed herein are either commercially available, publicly available on an unrestricted basis, or can be constructed from available plasmids by application of procedures known in the art. Many plasmids and other cloning and expression vectors that can be used in accordance with the present invention are well-known and readily available to those of skill in the art. Moreover, those of skill readily can construct any number of other plasmids suitable for use in the invention. The properties, construction and use of such plasmids, as well as other vectors, in the present invention will be readily apparent to those of skill from the present disclosure.

The term “expression vector” refers to a vector which comprises regulatory sequences necessary for transcription and translation of a cloned gene or genes and thus can transcribe and clone DNA. An expression vector can include one or more regulatory sequences, which can be selected based on the type of host cells used, operably linked to a gene. Regulatory sequences include promoters, enhancers and other expression control elements, for example, poly (A)+ sequences. Other expression vector components can include, but are not limited to, one or more of the following: a signal sequence, an origin of replication, one or more selection genes and a transcription termination sequence.

“Sequence” means the linear order in which monomers occur in a polymer, for example, the order of amino acids in a polypeptide or the order of nucleotides in a polynucleotide.

“Sequence identity or similarity”, as known in the art, is the relationship between two or more polypeptide sequences or two or more polynucleotide sequences, as determined by comparing the sequences. As used herein, “identity”, in the context of the relationship between two or more nucleic acid sequences or two or more polypeptide sequences, refers to the percentage of nucleotide or amino acid residues, respectively, that are the same when the sequences are optimally aligned and analyzed. For the purposes of comparing a queried sequence against, for example, the amino acid sequence SEQ ID NO: 2, the queried sequence is optimally aligned with SEQ ID NO: 2 and the best local alignment over the entire length of SEQ ID NO: 2 (307 amino acids) is obtained.

Analysis of sequence identity can be carried out manually or using sequence comparison algorithms. For sequence comparison, typically one sequence acts as a reference sequence, to which a queried sequence is compared. When using a sequence comparison algorithm, test and reference sequences are input into a computer, sub-sequence coordinates are designated, if necessary, and sequence algorithm program parameters are designated.

Optimal alignment of sequences for comparison can be conducted, for example, by using the homology alignment algorithm of Needleman & Wunsch, J Mol. Biol., 48:443 (1970). Software for performing Needleman & Wunsch analyses is publicly available through the Institut Pasteur (France) Biological Software website: http://bioweb.pasteur.fr/seqanal/interfaces/needle.html. The NEEDLE program uses the Needleman-Wunsch global alignment algorithm to find the optimum alignment (including gaps) of two sequences when considering their entire length. The identity is calculated along with the percentage of identical matches between the two sequences over the reported aligned region, including any gaps in the length. Similarity scores are also provided wherein the similarity is calculated as the percentage of matches between the two sequences over the reported aligned region, including any gaps in the length. Standard comparisons utilize the EBLOSUM62 matrix for protein sequences and the EDNAFULL matrix for nucleotide sequences. The gap open penalty is the score taken away when a gap is created; the default setting using the gap open penalty is 10.0. For gap extension, a penalty is added to the standard gap penalty for each base or residue in the gap; the default setting is 0.5.

Hybridization can also be used as a test to indicate that two polynucleotides are substantially identical to each other. Polynucleotides that share a high degree of identity will hybridize to each other under stringent hybridization conditions. “Stringent hybridization conditions” has the meaning known in the art, as described in Sambrook et al., Molecular Cloning: A Laboratory Manual, Second Edition, Cold Spring Harbor Laboratory, Cold Spring Harbor, N.Y., (1989). An exemplary stringent hybridization condition comprises hybridization in 6× sodium chloride/sodium citrate (SSC) at about 45° C., followed by one or more washes in 0.2× SSC and 0.1% SDS at 50-65° C., depending upon the length over which the hybridizing polynucleotides share complementarity.

A “reporter gene” refers to a nucleic acid sequence that encodes a reporter gene product. As is known in the art, reporter gene products are typically easily detectable by standard methods. Exemplary suitable reporter genes include, but are not limited to, genes encoding luciferase (lux), β-galactosidase (lacZ), green fluorescent protein (GFP), chloramphenicol acetyltransferase (CAT), β-glucuronidase, neomycin phosphotransferase, and guanine xanthine phosphoribosyl-transferase proteins.

The term “capable of LDC activity” as used herein includes any enzyme that can cleave the peptide bond between the preceding amino acid and a C-terminal D-alanine residue. Enzymes which are capable of LDC activity can cleave naturally occurring, cloned, mutated, recombinantly expressed, chemically synthesized, chemically modified substrates or substrates substituted with amino acid analogs and are able to cleave the peptide bond between the preceding amino acid and a C-terminal D-alanine residue. For example, the enzyme PA LDC can cleave the peptide bond between substrates such as; 1)“Tetrapeptide Lys” L-Ala-γ-D-Glu-L-Lys-D-Ala, 2) “Tetrapeptide DAP” L-Ala-γ-D-Glu-Diaminopimelic acid-D-Ala, and 3) “UDP-MurNAc-tetrapeptide” UDP-MurNAc-L-Ala-γ-D-Glu-Diaminopimelic acid-D-Ala, as described in Example 2. This activity can be detected by reverse phase HPLC or using the methods and kits of the present invention.

The term “PA-LDC enzyme activating temperature” as used herein refers to any temperature at which PA-LDC is active or can be activated to cleave the peptide bond between the preceding amino acid and a C-terminal D-alanine residue. For example a PA-LDC enzyme activating temperature can be 37 degrees Celsius. PA-LDC can be active at other temperatures as well, for example preferably between 35-40 degrees Celsius.

The terms “substrate” and “suitable substrate” as used herein mean any peptide or protein which is either naturally occurring, cloned, mutated, recombinantly expressed, chemically synthesized, chemically modified or substituted with amino acid analogs, that can be cleaved by an LDC. For example substrates include, but are not limited to, 1) “Tetrapeptide Lys” L-Ala-γ-D-Glu-L-Lys-D-Ala, 2) “Tetrapeptide DAP” L-Ala-γ-D-Glu-Diaminopimelic acid-D-Ala, and 3) “UDP-MurNAc-tetrapeptide” UDP-MurNAc-L-Ala-γ-D-Glu-Diaminopimelic acid-D-Ala, as described and employed in Example 2.

The term “epitope” as used herein refers a site on a large molecule against which an antibody will be produced and to which it will bind. Epitopes can be naturally occurring, chemically or synthetically produced. Epitopes to Class I MHC have an optimal size of 9 amino acids with a range from about 8 amino acids to about 11 amino acids. Epitopes which bind to Class II MHC have an optimal size range from about 12 amino acids to about 25 amino acids amino acids, however longer polypeptides can be used to generate antibodies if desired.

A “compound that decreases the enzymatic activity of an LDC enzyme” includes any compound that results in decreased cleavage of the substrate. In one embodiment, such a compound can decrease the rate of cleavage of the substrate. In another embodiment, such a compound can decrease the quantity of substrate cleaved. In another embodiment the compound can decrease the rate of cleavage of the substrate and the quantity of substrate cleaved.

In practicing the present invention, many conventional techniques in molecular biology, microbiology and recombinant DNA are used. These techniques are well-known and are explained in, for example, Current Protocols in Molecular Biology, Vols. I, II, and III, F. M. Ausubel, ed. (1997); and Sambrook et al., Molecular Cloning: A Laboratory Manual, Cold Spring Harbor Laboratory Press, Cold Spring Harbor, N.Y. (2001).

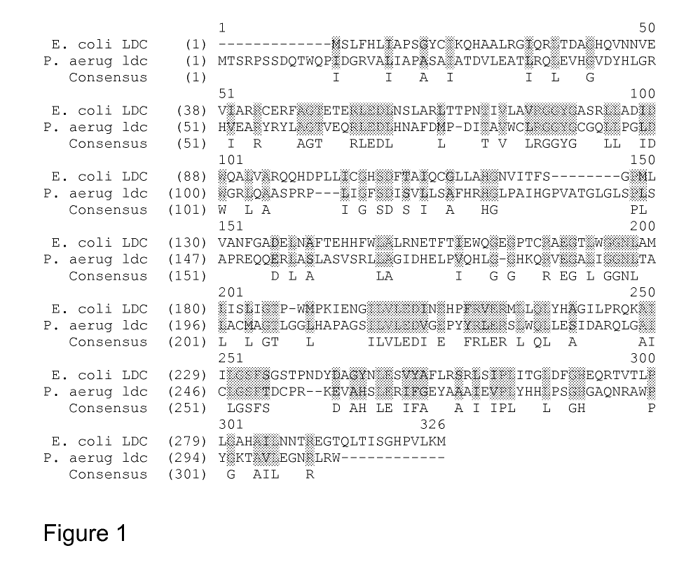

A putative homolog of LDC from P. aeruginosa (SEQ ID NO: 1) has now been cloned. The LDC of P. aeruginosa provides a means for identification of LDC inhibitors with potential utility for treating infections such as those caused by biofilms. An alignment of the E. coli LDC protein (muramoyl-tetrapeptide carboxypeptidase P76008, classified as peptidase with an unknown catalytic mechanism) with other bacterial proteins which are members of the U61 family was found in the MEROPS Peptidase Database (http://merops.sanger.ac.uk/). The putative protein of the present invention, PA5198 (SEQ ID NO:2) from P. aeruginosa was designated by MEROPS as an “unassigned peptidase” of the U61 family. The amino acid sequences of these two proteins were then aligned using the Vector NTI program (InforMax, Inc. Invitrogen Corp., Carlsbad, Calif.), which indicated a 25% identity and a 35% similarity (the latter accepts similar amino acids at a given position) in comparing the two proteins (FIG. 1). Based on these homologies it was predicted that this protein, PA5198 (SEQ ID NO:2) would function as an LDC, despite the fact that it shared such low homology with the E. coli LDC.

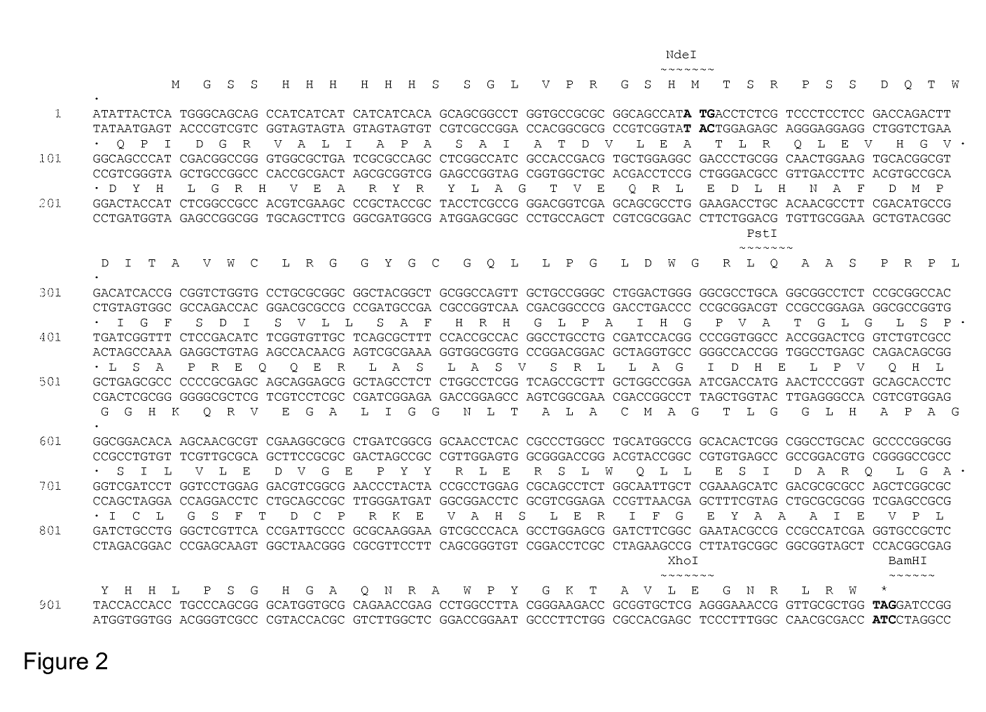

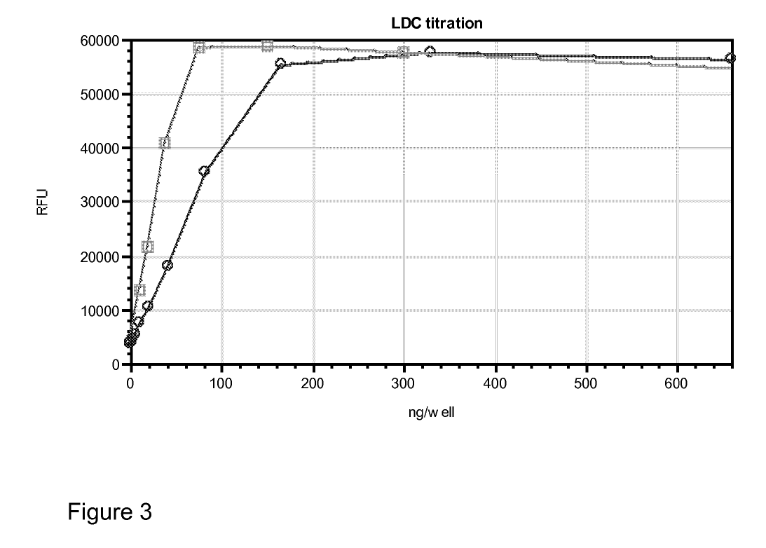

The P. aeruginosa open reading frame AAG08583.1 from strain PA01 (FIG. 2) was amplified by polymerase chain reaction using the primers depicted in SEQ ID NO: 3 and SEQ ID NO: 4 and cloned into the NdeI/BamHI sites of the N-terminal his-tagged protein expression vector pET14b (Novagen, EMD Biosciences, Madison, Wis.). DNA sequence analysis (ACGT, Inc. Wheeling, Ill.) confirmed the complete identity of the cloned gene (henceforth called PA LDC) with the Pseudomonas aeruginosa strain PA01 open reading frame accession number AAG08583.1 sequence in Genbank for a conserved hypothetical protein. U.S. Pat. No. 6,551,795, to Genome Therapeutics Corporation, discloses a SEQ ID NO: 22646 that is 100% identical to the GenBank sequence. The patent also discloses the DNA SEQ ID NO: 6075 that encodes the full length of the protein, however the applicants did not clone, express or purify the protein, nor have they shown utility for the expressed protein.

The present invention relates to novel P. aeruginosa LDC (PA LDC) nucleic acids, polypeptides and proteins encoded by these nucleic acids, recombinant PA LDC materials, and methods involving the production, detection, and utilization of these materials.

In attempts to clone the PA LDC homologue, a PCR-based strategy was employed. Oligonucleotide primers were synthesized according to the sequences set forth in SEQ ID NO: 3 (Forward PCR Primer PA_up 1) and SEQ ID NO: 4 (Reverse PCR Primer PA_down 1). These primers were able to successfully amplify a portion of the PA LDC sequence from position 1 to position 993 of SEQ ID NO: 1. In the present invention, the PA LDC gene was cloned from genomic DNA of P. aeruginosa strain PA01. The PA LDC gene was sequenced, and is shown as SEQ ID NO: 1 (FIG. 1). SEQ ID NO: 1 encodes a 307 residue polypeptide (SEQ ID NO: 2), also shown in FIG. 1, which is aligned with the LDC protein sequence from E. coli. By alignment, the cloned PA LDC polypeptide sequence (SEQ ID NO: 2) shares 77 (as indicated by consensus amino acid) out of 307 residues (25% identity). In the present invention, the PA LDC nucleic acid was also subcloned into an expression vector and transformed into a host cell for expression of the PA LDC protein. This recombinant PA LDC cell system was shown to express a functional PA LDC protein that cleaved a tetrapeptide substrate in a dose dependent manner.

The invention also relates to isolated nucleic acid fragments. Isolated nucleic acids comprising fragments of SEQ ID NO: 1 are useful for a variety of purposes. For example, these sequences can be used as oligonucleotide probes for the detection of LDC nucleic acids or for the detection of sequences that flank LDC nucleic acids. They can be used as oligonucleotide primers for the amplification of LDC nucleic acids. For many methods, oligonucleotides of about 16-25 nucleotides, or from about 26-35 nucleotides in length are useful, although longer oligonucleotides of greater than about 35 nucleotides can also be utilized. Some oligonucleotides can be used as “primers” for the synthesis of complimentary nucleic acid strands. For example, DNA oligonucleotide primers can hybridize to a complimentary nucleic acid sequence to prime the synthesis of a complimentary DNA strand in reactions using DNA polymerases. Oligonucleotides are also useful for hybridization in several methods of nucleic acid detection, for example, in Northern blotting or in situ hybridization. They can also be used for the preparation of chimeric nucleic acids that encode a portion or all of the PA LDC polypeptide fused to another polypeptide sequence, for example, one or more motifs or domains of the PA LDC sequence recombined with one or more motifs or domains from one or more heterologous sequences. In some embodiments this can affect the activity of the LDC enzyme.

In addition to nucleic acid sequences encoding PA LDC polypeptides, the invention also includes PA LDC polypeptides, PA LDC polypeptide variants, fragments of PA LDC polypeptides and PA LDC polypeptides having additional amino acids.

Inhibition of the function or expression of LDC proteins can be advantageous for the treatment of various infections. Since PA LDC is required for the stationary phase of the bacterial growth, it is anticipated that inhibition of PA LDC activity is also relevant for therapeutic applications where antibiotic treatment is used as a method of treating a bacterial infection, such as dental carries, periodontontitis, otitis media, musculosekletal infections, necrotizing fasciitis, biliary tract infection, osteomyelitis, bacterial prostatitis, native valve endocarditis, cystic fibrosis pneumonia, or meloidosis. (Costerton, et. al. 1999). For example, inhibition of the function or expression of LDC proteins can be useful in treating patients having bacterial biofilm infections, from species such as Acidogenic Gram-positive cocci, Gram-negative anaerobic oral bacteria, nontypable strains of Haemophilus influenzae, Gram-positive cocci (e.g., staphylococci), Group-A streptococci, enteric bacteria (e.g., Escherichia coli), Viridans group streptococci, Pseudomonas aeruginosa and Burkholderia cepacia and Pseudomonas pseudomallei. Inhibition of LDC activity can also be relevant in patients suffering from nosocomial infections or diseases related to intensive care unit pneumonia, sutures, exit sites, arterioveneous shunts, schleral buckles, contact lenses, urinary catheter cystitis, peritoneal dialysis, continuous ambulatory peritoneal dialysis (CAPD), peritonitis, intrauterine devices, endotracheal tubes, Hickman catheters, central venous catheters, mechanical heart valves, vascular grafts, biliary stent blockages, orthopedic devices, and penile prostheses. The preceding infections or diseases can be caused by biofilm bacterial species such as Gram-negative rods, Gram-positive cocci, Staphylococcus epidermidis and S. aureus, Pseudomonas aeruginosa, and Actinomyces israelii. See Costerton, J. W., Stewart. P. S., and Greenberg, E. P. Bacterial biofilms: a common cause of persistent infections. Science 284, 1318-1322 (1999).

PA LDC polypeptide variants are polypeptides capable of LDC activity in which substitutions have been made in the amino acid sequence from the sequence shown in SEQ ID NO:2. These substitutions can be as a result of naturally occurring mutations during DNA synthesis, transcription or translation, the use of amino acid analogs, chemical modifications or other molecular biology techniques well known in the art.

Fragments of PA LDC polypeptides include polypeptides capable of LDC activity which are shorter in length than the sequence shown in SEQ ID NO:2. These fragments include any polypeptide capable of LDC activity that is less than 307 amino acids in length. For example a fragment of PA LDC can be from 306 to about 200 amino acids in length and still be capable of LDC activity. Fragments of PA LDC polypeptides may be created by naturally occurring mutations during DNA synthesis, transcription or translation. Those skilled in the art will readily recognize that the use of enzymes which cleave either DNA or proteins, other molecular biology techniques or chemical modifications can be employed to create fragments of PA LDC polypeptides. For example, a restriction endonuclease can be employed to cleave DNA at a specific site to create fragments of the PA LDC polypeptides. In another example, chemical cleavage of polynucleotides can be accomplished using glutathione in the presence of copper ions.

PA LDC polypeptides or PA LDC polypeptide fragments can be generated using a variety of synthetic or molecular biological technique. Standard synthetic peptide techniques can be used to generate smaller PA LDC polypeptide fragments, for example peptide fragments that are 30 amino acids in length or shorter. Techniques for the synthesis of peptides fragments are well known and are described in, for example, Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al., J. Am. Chem. Soc., 85: 2149-2156 (1963), and Stewart et al., Solid Phase Peptide Synthesis, 2nd ed. Pierce Chem. Co., Rockford, Ill. (1984).

PA LDC polypeptides having additional amino acids are also envisioned within the scope of the invention which are longer in length than the sequence shown in SEQ ID NO:2. As described herein, the PA LDC polypeptide can also have additional amino acid residues at its amino terminus, its carboxyl terminus or both. Such additional residues are useful for a variety or purposes, including, for example, immunodetection, purification, cellular trafficking, enzymatic activity, etc. These proteins can include any polypeptide capable of LDC activity that is greater than 307 amino acids in length. For example a recombinant PA LDC can be from 308 to about 400 amino acids in length and still be capable of LDC activity. These PA LDC polypeptides can be created by naturally occurring mutations during DNA synthesis, transcription or translation, the use of enzymes that ligate either DNA (such as T4 ligase) or fuse proteins. For example, during DNA synthesis and RNA transcription, extra nucleotides can be added to the extending polynucleotide as a result of mutation. Additionally, enzymes such as T4 ligase can be employed to ligate polynucleotides together. During the process of translation, mutations such as insertions and fusions can create a polypeptide which is longer than the protein encoded by a DNA sequence.

Those skilled in the art will readily recognize that the use of other molecular biology techniques can be employed to create recombinant proteins or protein fusions.

Additionally fragments of PA LDC and PA LDC polypeptides having additional amino acids can comprise variant sequences in which substitutions have been made in the amino acid sequence using the sequence shown in SEQ ID NO:2. These substitutions can be as a result of naturally occurring mutations during DNA synthesis, transcription or translation, the use of amino acid analogs, chemical modifications or other molecular biology techniques well known in the art.

Due to the degeneracy of the genetic code, more than one codon can be used to encode a particular amino acid, and therefore, a PA LDC amino acid sequence (for example, SEQ ID NO: 2) can be encoded by any one of a plurality of nucleic acid sequences. Isolated nucleic acid includes sequences wherein one or more codons in the sequence are replaced by codons of a different sequence but that code for the same amino acid residue are herein referred to as “conservative codon substitutions”. Therefore, the invention encompasses nucleic acid sequences encoding SEQ ID NO: 2 that have one or more than one conservative codon substitution. One of skill in the art would be able to determine a particular nucleic acid sequence having one or more than one conservative codon substitution and encoding SEQ ID NO: 2, based on the sequence information provided herein. Conservative codon substitutions can be made in the nucleic acid sequence encoding the LDC polypeptide, for example, the codons TTT and TTC (collectively referred to as TTT/C) can encode a Phe (phenylalanine) residue; other codon substitutions are as follows: TTA/G and CTT/C/A/G: Leu; ATT/C: Ile; ATG: Met; GTT/C/A/G: Val; TCT/C/A/G: Ser; CCT/C/A/G: Pro; ACT/C/A/G: Thr; GCT/C/A/G: Ala; TAT/C: Tyr; CAT/C: His; CAA/G: Gln; AAT/C: Asn; AAA/G: Lys; GAT/C: Asp; GAA/G Glu; TGT/C: Cys; CGT/C/A/G: Arg; AGT/C: Ser; AGA/G; Arg; GGT/C/A/G:Gly. Conservative codon substitutions can be made at any position in the nucleic acid sequence that encodes the PA LDC polypeptide while still preserving LDC activity.

Based on this analysis, a PA LDC polypeptide having a substitution of one or more LDC-family variant amino acids is anticipated to have LDC biological activity. That is, SEQ ID NO: 2 can be substituted at one or more LDC-family variant amino acid positions with an amino acid selected from amino acid residues found in the E. coli sequence, or an equivalent amino acid, at that same position. The amino acid that replaces a PA LDC amino acid is herein referred to as a “LDC-family variant amino acid”. A “LDC-family variant amino acid” consists of an amino acid that differs from the PA LDC amino acid and that is the amino acid present in the LDC sequence of other bacteria, such as E. coli. For example, an LDC-family variant amino acid at position 50 suitable for the replacement of the arginine (R) of SEQ ID NO: 2, is glutamic acid (E), as occurs in E. coli LDC based upon the alignment shown in FIG. 1.

At some LDC-family variant amino acid positions, the P. aeruginosa and the E. coli acid residues share a common chemical property. For example, LDC-family variant amino acids at positions 112 and 129 of SEQ ID NO: 2 can include a hydrophobic amino acid residue, for example, isoleucine (I) or leucine (L). Other hydrophobic amino acids include glycine, valine, methioinine and proline. Other amino acid groups include “basic amino acids,” which include histidine, lysine, and arginine; “acidic amino acids,” which include glutamic acid and aspartic acid; “aromatic amino acids,” which include phenylalanine, tryptophan, and tyrosine; “small amino acids,” which include glycine and alanine; “nucleophilic amino acids,” which include serine, threonine, and cysteine; and “amide amino acids,” which include aparagine and glutamine. Amino acid substitutions can therefore be made in recombinant LDC polypeptides while retaining the enzyme's carboxypeptidase activity, by selecting those amino acid which residues share a common chemical property between P. aeruginosa and the E. coli at any given position.

In another aspect, the invention provides a nucleic acid encoding a LDC polypeptide according to SEQ ID NO: 2 that includes one or more LDC-family variant amino acids. In some embodiments, PA LDC polypeptides include LDC-family variant amino acids in less than 25% of the original PA LDC amino acid residues. Preferably the PA LDC polypeptides include LDC-family variants in less than about 20% of the original PA LDC amino acid residues, and most preferably less than about 15% of the original PA LDC amino acid residues. The invention also provides isolated nucleic acid molecules that are complementary to any isolated nucleic acid molecules, as described herein.

The isolated nucleic acid of the invention can also include nucleic acid sequences that encode the PA LDC polypeptide having additional amino acid residues. In some embodiments, the additional amino acids are present at the amino terminus, the carboxyl terminus, within the PA LDC sequence or combinations of these locations. PA LDC polypeptides having these types of additional amino acid sequences can be referred to as “PA LDC fusion proteins”. In some cases, it may be more appropriate to refer to them otherwise as “chimeric” or “tagged” PA LDC proteins, or the like, depending on the nature of the additional amino acid sequences. Nonetheless, one will be able to discern a LDC polypeptide having additional amino acid sequences given the sequence information provided herein. The additional amino acid residues can be short, for example, from one to about 20 additional amino acid residues, or longer, for example, greater than about 20 additional amino acid residues. The additional amino acid residues can serve one or more functions or purposes including, for example, serving as epitopes for protein (e.g., antibody) or small molecule binding; serving as tags for intracellular and extracellular trafficking; providing additional enzymatic or other activity; or providing a detectable signal.

Recombinant techniques can be used for the expression of PA LDC including but not limited to fragments of PA LDC, variants of PA LDC and fusions of PA LDC with other proteins from host cells transformed with a PA LDC nucleic acid. These methods include, for example, in vitro recombinant DNA techniques and in vivo genetic recombination (see, for example, the techniques described in Sambrook et al., Molecular Cloning, A Laboratory Manual, 3'd Edition, Cold Spring Harbor Press, N.Y. (2001); and Ausubel et al., eds., Short Protocols in Molecular Biology, 4th Edition, John Wiley & Sons, Inc., NY (1999)).

PA LDC protein can be produced by introducing an expression vector encoding a PA LDC polypeptide into a cell and culturing the cells to express the polypeptide. When a purified PA LDC polypeptide is desired, a step can also be performed to isolate and, if desired, purify the PA LDC polypeptide.

In another embodiment, the invention provides a recombinant nucleic acid construct that includes the entire or a portion of the PA LDC coding sequence operably linked to a regulatory sequence. These recombinant nucleic acid constructs include recombinant expression vectors suitable for expression of the PA LDC nucleic acid in a host cell. Recombinant expression vectors include one or more regulatory sequences, which can be selected based on the type of host cells used for PA LDC expression, operably linked to a PA LDC nucleic acid sequence. Regulatory sequences include promoters, enhancers and other expression control elements, for example, poly (A)+ sequences. Regulatory sequences can be specific for prokaryotic cells, for example, bacterial cells, such as E. coli, or for eukaryotic cells, such as yeast cells, insect cells or mammalian cells (for example, HEK, CHO or COS cells). Regulatory sequences can be located cis or trans relative to the PA LDC nucleic acid sequence. Regulatory sequences can include constitutive expression sequences that typically drive expression of the nucleic acid under a wide variety of growth conditions and in a wide variety of host cells, tissue-specific regulatory sequences that drive expression in particular host cells or tissues and inducible regulatory sequences that drive expression in response to a secondary factor. Choice and design of the expression vector can depend on such factors as the particular host cell utilized and the desired levels of polypeptide expression. Other expression vector components can include, but are not limited to, one or more of the following: a signal sequence, an origin of replication, one or more genes that facilitate selection of a transformed cell or nucleic acid from the transformed cell and a transcription termination sequence. Genes facilitating selection of transformed cells encode proteins that (a) confer resistance to antibiotics or other toxins, for example, ampicillin, neomycin, methotrexate or tetracycline, (b) complement auxotrophic deficiencies or (c) supply critical nutrients not available from complex media.

Recombinant nucleic acid constructs used for expression of the PA LDC polypeptide can also include constructs that can be transcribed and translated in vitro, for example, constructs having a T7 promoter regulatory sequence.

Vectors suitable for the expression of PA LDC are known in the art and commercially available. Suitable vectors include, for example, pET-14b, pCDNAlAmp and pVL1392, which are available from Novagen and Invitrogen and can be used for expression in E. coli, COS cells and baculovirus infected insect cells, respectively.

In another embodiment, the invention provides a recombinant cell that includes a PA LDC nucleic acid. Recombinant cells include those wherein a nucleic acid sequence has been introduced. Typically, recombinant cells are created by introducing a particular nucleic acid into cells using molecular biological techniques. However, recombinant cells also include cells that have been manipulated in other ways to promote the expression of a desired nucleic acid sequence. For example, regions that are proximal to a target nucleic acid sequence can be altered to promote expression of the target nucleic acid, or genes that act to regulate the expression of a target nucleic acid can be introduced into a cell.

Recombinant cells, after periods of growth and division, may not be identical to the starting parent cell; however, these cells are still referred to as recombinant cells and are included within the scope of the term as used herein.

Host cells suitable for harboring and providing the machinery for PA LDC expression include any prokaryotic cells. Examples of suitable prokaryotic host cells are eubacteria, such as Gram-negative or Gram-positive organisms, for example, Enterobacteriaceae such as Escherichia, for example, E. coli, Enterobacter, Salmonella, for example, Salmonella typhimurium, as well as Bacilli such as B. subtilis, Pseudomonas, and Streptomyces.

Growth of the transformed host cells can occur under conditions that are known in the art. The conditions will generally depend upon the host cell and the type of vector used. Suitable induction conditions, such as temperature and chemicals, can be used and will depend on the type of promoter utilized. Examples of suitable media for the propagation of prokaryotes include LB, Luria broth (LB), also known as Miller's L Broth.

Nucleic acids, including expression constructs, can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, virus mediated introduction, biolistics or electroporation. Suitable methods for transforming or transfecting host cells can be found in Sambrook, et al. (supra), and other laboratory manuals. Recombinant cells can be useful for the production of a PA LDC polypeptide for purification purposes or for functional studies involving the PA LDC polypeptide. For example, a recombinant PA LDC cell can be used in assays to test a number of compounds for their ability to alter the activity of the PA LDC polypeptide. The recombinant PA LDC cell can also be used in assays to test how altering various properties of the PA LDC polypeptide, for example, altering the amino acid sequence of the PA LDC polypeptide, affects PA LDC activity.

A variety of methods can be used for purification of the PA LDC polypeptide. For example, crude purification can be performed using ammonium sulfate precipitation, centrifugation or other known techniques. A higher degree of purification can be achieved by suitable chromatographic techniques, including, for example, anion exchange, cation exchange, high performance liquid chromatography (HPLC), gel filtration, hydrophobic interaction chromatography and affinity chromatography, for example, as well as immunoaffinity chromatography using antibodies directed against the PA LDC protein. If needed, steps for refolding the PA LDC proteins can be used to obtain the active conformation of the protein when the protein is denatured during intracellular synthesis. For example, isolation or purification of the protein by resuspending the protein in 6M urea and subsequent dialyzation of the urea can be used to refold the protein.

A nucleic acid molecule can encode a PA LDC fusion protein, which can include additional amino acid residues that provide coordinates for bonding such as ionic, covalent, hydrogen or Van der Waals bonding or combinations thereof with organic or inorganic compounds. Useful additional amino acid fragments include, for example, poly-histidine residues useful for protein purification using Ni⁺-coupled residues, constant domains of immunoglobulins (IgA, IgE, IgG, IgM) or portions thereof (CH1, CH2, CH3), albumin, hemagluttinin (HA) or myc affinity epitope tags useful for the formation of immuno-complexes for detection or purification (antibodies against these moieties can be obtained commercially), polypeptides useful for detection such as the green fluorescent protein (GFP), enzymes such as beta-galactosidase (B-Gal) chloramphenicol acetyltransferase (CAT), luciferase, and alkaline phosphatase (A), signal sequences for protein trafficking and protease cleavage sequences useful for separating additional amino acid sequences from the PA LDC sequence, if desired.

In another aspect, diagnostic assays are provided which are capable of detecting the expression of a PA LDC protein or nucleic acid. Expression of the PA LDC proteins can be detected by a probe capable of binding to the PA LDC protein. Preferably the probe is detectably labeled or labeled subsequent to probe binding to the PA LDC protein. Typically, the probe is an antibody that recognizes the expressed protein, as described above, especially a monoclonal antibody. Accordingly, in one embodiment, an assay capable of detecting the expression of PA LDC protein comprises contacting a biological sample with one or more than one monoclonal and/or polyclonal antibody that binds to PA LDC and detecting the antibody bound to the PA LDC.

In one aspect, the present invention relates to antibodies that specifically recognize epitopes within the amino acid sequence of SEQ ID NO: 2. For example antibodies which recognize epitopes selected from polypeptides ranging in length from as little as 15 amino acid peptides to larger peptides including any polypeptide including at least about 15 consecutive amino acids of SEQ ID NO:2.

Useful antibodies include, but are not limited to, polyclonal antibodies, monoclonal antibodies, humanized or chimeric antibodies, and biologically functional antibody fragments that are able to bind to a portion of the PA LDC protein. Antibodies specific for proteins encoded by the aforementioned sequences have utilities in several types of applications. These antibodies can be used in diagnostic kits, for example, in a variety of assays wherein detection of PA LDC is desired. They can also be used in the preparation of therapeutic agents, for example, wherein the anti-PA LDC antibody itself is therapeutic or wherein the anti-PA LDC antibody is coupled to a therapeutic agent.

The invention also provides methods for the production of human-specific monoclonal anti-PA LDC antibodies. For the production of these monoclonal antibodies, peptides that provide unique anti-PA LDC determinants can be used. Monoclonal antibodies are homogeneous clonal populations of antibodies that are directed to a specific antigen (i.e., epitope). To prepare anti-PA LDC monoclonal antibodies, a peptide having a PA LDC-specific sequence or a “PA LDC epitope” is used. A PA LDC sequence is one that is different at one or more positions relative to the LDC sequences of other species. In order to determine a PA LDC specific sequence, one can refer to FIG. 2 or SEQ ID NO: 1 provided herein.

For the production of antibodies to the LDC protein, various host animals can be immunized by injection with the PA LDC polypeptide, or a portion thereof. If the entire PA LDC polypeptide is used, antibodies specific to PA LDC along with anti-CMR antibodies that are cross-reactive with other LDC proteins from different species can be generated. Where a particular peptide of the PA LDC protein is to be used for the production of an antibody, one or more peptides or polypeptides of a desired length can be used to produce antibody. Those of ordinary skill in the art of antibody production will recognize that antibody can be isolated from the sera of animals exposed to one or more peptides or polypeptides ranging in length from as little as 15 amino acid peptides to larger peptides including any polypeptide including at least about 15 consecutive amino acids of SEQ ID NO:2. For example, polyclonal antibody preparations comprise a heterogeneous population of antibody molecules derived from the sera of animals immunized with full length, peptides or polypeptides of the PA LDC protein. In this polyclonal population, antibodies will be cross-reactive with different portions of the LDC polypeptide, with some of those antibodies being specifically reactive with PA LDC and others being cross-reactive with LDC polypeptides of other species. For the production of polyclonal antibodies, host animals are immunized with the PA LDC protein, or one or more portions thereof, typically repeatedly to boost antibody titer in the animal and typically supplemented with adjuvants as described herein. Commonly used host animals for the production of anti-LDC antibodies include rabbits, mice and rats; however, other animals can be used if desired. Various adjuvants can be used to increase the immunological response, depending on the host species, for example, Freund's (complete and incomplete) adjuvant and mineral gels such as aluminum hydroxide. Conjugates (e.g., KLH) can also be included for the immunization, especially in cases where shorter PA LDC peptides are used for the purposes of immunization and antibody production.

Monoclonal antibodies (mAbs) can be obtained by any technique that provides for the production of antibody molecules by continuous cell lines in culture. These include, for example, the hybridoma technique (Kohler and Milstein, Nature, 256:495-497, 1975); the human B-cell hybridoma technique (Kosbor et al., Immunology Today, 4:72, 1983); and the EBV-hybridoma technique (Cole et al., Monoclonal Antibodies and Cancer Therapy, Alan R. Liss, Inc., pp. 77-96, 1985). Such antibodies can be of any immunoglobulin class including IgG, IgM, IgE, IgA, IgD or any subclass thereof. The hybridoma producing the mAb of this invention can be cultivated in vitro or in vivo.

Immunoassay methods that utilize antibodies include, but are not limited to, dot blotting, Western blotting, competitive and non-competitive protein binding assays, enzyme-linked immunosorbant assays (ELISA), immunohistochemistry, fluorescence-activated cell sorting (FACS), immuno-PCR, immunoprecipitation and others commonly used.

In other diagnostic assays which are capable of detecting the expression of PA LDC, the level of expression of mRNA corresponding to the PA LDC gene can be detected utilizing commonly used molecular biological methods, for example, Northern blotting, in situ hybridization, nuclease protection assays, RT-PCR (including real-time, quantitative PCR), high density arrays and other hybridization methods. Accordingly, in another embodiment, an assay capable of detecting the expression of one or more than one PA LDC gene in a biological sample is provided, which comprises contacting a biological sample with an oligonucleotide capable of hybridizing to a PA LDC nucleic acid. The oligonucleotide is generally from 10-20 nucleotides in length for PCR/primer extension experiments. Longer oligonucleotides of approximately 40-50 nucleotides are more regularly utilized for in situ or blot hybridizations. Sequences even longer than 50 nucleotides can also be employed in the detection methods of this invention. RNA can be isolated from the tissue sample by methods well-known to those skilled in the art as described, for example, in Ausubel et al., Current Protocols in Molecular Biology, John Wiley and Sons, Inc. (1996). One preferred method for detecting the level of mRNA transcribed from the PA LDC genes is by RT-PCR. Details of RT-PCR techniques are well known and also described, for example, in Pfeffer et.al. Efficient one-tube RT-PCR amplification of rare transcripts using short sequence-specific reverse transcription primers. BioTechniques 18, 204-206. (1995) and Giannoni et al. An improved reverse transcription-polymerase chain reaction method to study a lipoprotein gene. Journal of Lipid Research 35 (2): 340. (1994).

Another preferred method for detecting the level of mRNA transcripts obtained from the disclosed genes of this invention involves hybridization of labeled mRNA to an ordered array of oligonucleotides or tissue. Such a method allows the level of transcription of a plurality of these genes to be determined simultaneously to generate gene expression profiles or patterns.

The oligonucleotides utilized in this hybridization method typically are bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc. Any solid surface to which the oligonucleotides can be bound, either directly or indirectly, either covalently or noncovalently, can be used. A particularly preferred solid substrate is a high-density array or DNA chip. These high-density arrays contain a particular oligonucleotide probe in a pre-selected location on the array. Each pre-selected location can contain more than one molecule of the particular probe. Because the oligonucleotides are at specified locations on the substrate, the hybridization patterns and intensities (which together result in a unique expression profile or pattern) can be interpreted in terms of expression levels of particular genes.

The oligonucleotide probes are preferably of sufficient length to specifically hybridize only to complementary transcripts of the above identified gene(s) of interest.

Optionally, all or a portion of the PA LDC nucleic acid sequence can be used to probe nucleic acid preparations from other species to determine the presence of similar sequences. For example, all or a portion of the PA LDC nucleic acid can be used as a probe to identify cDNA or genomic nucleic acid sequences from other species that are similar to the PA LDC sequence, for example from a variety of strains of Pseudomonas. Positive clones can be identified as those that hybridize to the PA LDC probe.

In addition, all or a portion of the PA LDC nucleic acid or polypeptide sequence as provided by the invention can be used in computer-aided programs to identify other useful information, for example, proteins having homology to the PA LDC sequence, molecules that bind to the PA LDC sequence or molecules that are cleaved by the PA LDC polypeptide. For example, all or portions of the PA LDC sequence can be used to screen various electronic databases to determine whether a member of the electronic database has homology to the PA LDC sequence. Numerous genetic databases that are species-specific can be queried using any portion of the PA LDC nucleic acid or polypeptide sequences as set forth herein. Either or both nucleic acid and protein searches can be performed.

In additional methods, PA LDC nucleic acids and proteins, antibodies directed against LDC and biological systems containing any of these components can be labeled with a detectable reagent. Furthermore, a compound having specificity for PA LDC can be labeled with a detectable reagent and used to detect the PA LDC entity. Detectable reagents include compounds and compositions that can be detected by spectroscopic, biochemical, photochemical, bioelectronic, immunochemical, electrical, optical or chemical techniques. Examples of detectable moieties include, but are not limited to, radioisotopes (e.g., ³²P ³³P, ³⁵S), chemiluminescent compounds, labeled binding proteins, heavy metal atoms, spectroscopic markers, such as fluorescent markers and dyes, linked enzymes, mass spectrometry tags and magnetic labels.

In another aspect, a three-dimensional model of the PA LDC polypeptide can be determined and used to identify molecules that bind to various portions of the protein structure. For example, using an isolated PA LDC nucleic acid as described herein, the PA LDC protein can be expressed in a cell system, purified and then crystallized in order to obtain information regarding the structure of the protein. Structural information can be obtained by performing, for example, X-ray diffraction or nuclear magnetic resonance spectroscopy. The location of amino acid residues and their side chains can be expressed as coordinates in a three-dimensional model. This information can then be provided to a computer program.

Molecular modeling programs can be used to determine whether a small molecule can fit into a functionally relevant portion, for example, an active site, of the PA LDC polypeptide. Basic information on molecular modeling is provided in, for example, M. Schlecht, Molecular Modeling on the PC, 1998, John Wiley & Sons; Gans et al., Fundamental Principals of Molecular Modeling, 1996, Plenum Pub. Corp.; N. C. Cohen (editor), Guidebook on Molecular Modeling in Drug Design, 1996, Academic Press; and W. B. Smith, Introduction to Theoretical Organic Chemistry and Molecular Modeling, 1996. U.S. Patents that provide detailed information on molecular modeling include U.S. Pat. Nos. 6,093,573; 6,080,576; 5,612,894; and 5,583,973.

Programs that can be useful for molecular modeling studies include, for example, GRID (Goodford, P. J., “A Computational Procedure for Determining Energetically Favorable Binding Sites on Biologically Important Macromolecules” J. Med. Chem., 28, pp. 849-857, 1985), available from Oxford University, Oxford, UK; MCSS (Miranker, A. and M. Karplus, “Functionality Maps of Binding Sites: A Multiple Copy Simultaneous Search Method.” Proteins: Structure, Function and Genetics, 11, pp. 29-34, 1991), available from Molecular Simulations, Burlington, Mass.; AUTODOCK (Goodsell, D. S. and A. J. Olsen, “Automated Docking of Substrates to Proteins by Simulated Annealing” Proteins: Structure. Function, and Genetics, 8, pp. 195-202, 1990); available from Scripps Research Institute, La Jolla, Calif.; and DOCK (Kuntz, I. D. et al., “A Geometric Approach to Macromolecule-Ligand Interactions” J. Mol. Biol., 161, pp. 269-288, 1982), available from University of California, San Francisco, Calif.

The invention also encompasses kits for detecting the presence of a PA LDC polypeptide or nucleic acid of the invention in a biological sample (a test sample). Such a kit preferably comprises a compartmentalized carrier suitable to hold in close confinement at least one container. The carrier can contain a means for detection such as labeled antigen or enzyme substrates or the like. For example, the kit can comprise a labeled compound or agent capable of detecting the polypeptide or mRNA encoding the polypeptide and means for determining the amount of the polypeptide or mRNA in a sample (e.g., an antibody which binds the polypeptide or an oligonucleotide probe which binds to DNA or mRNA encoding the polypeptide).

For antibody-based kits, the kit can comprise, for example: (1) a first antibody (for example, an antibody attached to a solid support), which binds selectively to an epitope on the polypeptide comprising an amino acid sequence having at least 96% sequence identity to SEQ ID NO: 2; and, optionally; (2) a second antibody which binds to either the first antibody or a second epitope on the polypeptide, and which is conjugated to a detectable agent; and (3) optionally a purified recombinant PA LDC protein as a positive control. Preferably, one of the antibodies specifically binds to a PA LDC, and preferably does not bind to a carboxypeptidase from other species, such as E. coli, human, rat, or mouse.

For oligonucleotide-based kits, the kit can comprise, for example: (1) an oligonucleotide, e.g., a detectably labeled oligonucleotide, which hybridizes under stringent condition to SEQ ID NO: 1, or (2) a pair of oligonucleotide primers useful for amplifying a nucleic acid molecule encoding a polypeptide having at least 96% sequence identity to SEQ ID NO: 2. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample. Each component of the kit is usually enclosed within an individual container and all of the various containers are preferably contained within a single package.

Because LDC is required during the stationary phase of the bacterial lifecycle, this gene can serve as a therapeutic target for the identification of candidate compounds useful in treating bacterial infections, for example, detecting candidate compounds such as described herein. Therefore, in another general aspect, the present invention relates to the use of PA LDC nucleic acids and proteins in methods for identifying therapeutic compounds, for example, compounds useful in treating bacterial infections, during the stationary phase of the bacterial lifecycle. These types of compounds can be identified using a system that includes a PA LDC polypeptide or a PA LDC nucleic acid. Compounds can also be tested directly in vivo in an animal model system, for example, a rat, mouse or canine model system or directly in bacterial cultures. Particularly useful systems include animal models of stationary phase bacterial infections and bacterial models of biofilms. These methods comprise assaying for the ability of various compounds to increase or decrease the expression of the PA LDC protein or the enzymatic activity of the PA LDC protein.

The compound screening and identification methods can be performed using conventional laboratory formats or in assays adapted for high throughput. The term “high throughput” refers to an assay design that allows easy screening of multiple samples simultaneously and/or in rapid succession, and can include the capacity for robotic manipulation. Another desired feature of high throughput assays is an assay design that is optimized to reduce reagent usage, or minimize the number of manipulations in order to achieve the analysis desired. Examples of assay formats include 6-well, 24-well, 96-well or 384-well plates, levitating droplets, micro-array, macro-array and “lab on a chip” microenzyme chips used for liquid handling experiments. It is well known by those in the art that as miniaturization of plastic molds and liquid handling devices are advanced, or as improved assay devices are designed, greater numbers of samples can be processed using the design of the present invention.

Candidate compounds encompass numerous chemical classes, including but not limited to, small organic or inorganic compounds, natural or synthetic molecules, such as antibodies, proteins or fragments thereof, antisense nucleotides, interfering RNA (iRNA) and ribozymes. Preferably, the candidate compounds are small organic compounds, i.e., those having a molecular weight of more than 100 yet less than about 1000 Daltons. Candidate compounds comprise functional chemical groups necessary for structural interactions with polypeptides, and can include at least an amine, carbonyl, hydroxyl or carboxyl group, two of the functional chemical groups or three or more of the functional chemical groups. The candidate compounds can comprise cyclic carbon or heterocyclic structure and/or aromatic or polyaromatic structures substituted with one or more of the above-identified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound typically is a DNA or RNA molecule, although modified nucleic acids having non-natural bonds or subunits are also contemplated. Modified nucleic acids can comprise nucleoside analogs, nucleotide analogs, non-nucleoside analogs, non-nucleotide analogs and others.

Candidate compounds are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including biological libraries; spatially addressable parallel solid phase or solution phase libraries: synthetic library methods requiring deconvolution; the “one-bead one-compound” library method; and synthetic library methods using affinity chromatography selection (Lam (1997) Anticancer Drug Des. 12:145). Alternatively, libraries of natural compounds in the form of bacterial, fungal, plant and animal extracts are available or readily produced. Additionally, natural and synthetically produced libraries and compounds can be readily modified through conventional chemical, physical, and biochemical means.

In one aspect, the invention provides a method of identifying a compound that decreases the expression of a PA LDC protein, comprising the steps of: (a) contacting a test compound with a cell expressing PA LDC; and (b) determining whether the test compound decreases the expression PA LDC in the cell. Compounds identified by this mechanism can bind the PA LDC gene directly or indirectly. For example, compounds altering LDC expression include compounds that change those nuclear, cytoplasmic, or intracellular factors and influence the control of gene activity at the level of transcription or translation; such as gene repression. The cell comprising a PA LDC gene can be a native host cell that expresses PA LDC endogenously, such as a Pseudomonas auerginosa bacterial cell. The cell can also be a recombinant cell for example, a bacterial cell of another species, a yeast cell, an insect cell, or a mammalian cell containing a recombinant DNA sequence having a regulatory sequence for a PA LDC gene, where the regulatory sequence is operably linked to a gene, preferably a reporter gene.

The effect of the compound on the expression of PA LDC can be measured by a variety of means. For example, the effect can be measured by the amount of mRNA or protein of the gene from the cell, or by the activity of the gene product (the enzyme activity) from the cell. When a reporter gene is used, the effect can be measured as the level of reporter gene product from the cell. For example, when the LDC regulatory sequence is operably linked to a GFP gene, the effect of the compound on gene expression can be measured as the effect of the compound on emissions of green fluorescence from the cell using a fluorometer. When an endogenous PA LDC expressing cell is used, the effect of the compound on gene expression can be measured by the amount of PA LDC mRNA or protein inside the cell using methods described infra (i.e., Northern Blot, RT-PCR, SDS-PAGE, Western Blot, immunohisto- or immunocytochemistry, radioreceptor ligand binding, etc). Alternatively, the enzymatic activity of PA LDC enzyme can be used to measure the effect of the compound on the expression of the PA LDC protein as described infra.

The cell-based method described herein not only identifies compounds that regulate PA LDC expression directly via binding to one or more than one regulatory sequence of the PA LDC gene, but also identifies compounds that regulate PA LDC expression indirectly via binding to other cellular components whose activities influence PA LDC expression or protein stability. For example, compounds that regulate the activity of a transcriptional activator, inhibitor or other transcription factors for PA LDC genes can be identified using the method described herein. Compounds that regulate the activity of a protease that degrades the PA LDC enzyme in vivo can also be identified.

The invention also provides a method of identifying a compound that decreases the activity of PA LDC enzyme, comprising the steps of: (a) contacting the PA LDC protein with a substrate and a test compound; and (b) determining whether the test compound decreases the enzymatic activity of the PA LDC. In some embodiments, the PA LDC enzyme is expressed inside of a recombinant host cell. The cell can be a native host cell for PA LDC that expresses the PA LDC endogenously, for example, a Pseudomonas aeruginosa cell. The cell can also be a recombinant host cell for PA LDC, for example, a bacterial cell of another species, a yeast cell, an insect cell, or a mammalian cell expressing a PA LDC recombinantly.

The assays to identify a compound that decreases PA LDC enzyme activity are preferably performed under conditions in which the enzyme is known to be active. For example, such assays can be performed at an LDC activation temperature of 37 degrees Celsius and pH of 8.5. The pH can be varied from about 7.0 to about 9.0. Further, the incubation temperature can be varied from about 35 degrees Celsius to about 40 degrees Celsius.

Assays for the identification of PA LDC modulators can be carried out manually or using an automated system as described supra. Each well can contain Pseudomonas aeruginosa cells which express PA LDC endogenously. In another example the culture plates are filled with recombinant cells expressing a nucleic acid encoding the PA LDC protein, or a purified PA LDC protein is used. The LDC enzyme cleaves a suitable substrate, for example a tetrapeptide such as L-Ala-γ-D-Glu-L-Lys-D-Ala (Tetrapeptide Lys) and liberates a cleavage product such as D-Alanine. In other examples, suitable substrates can include peptides from about 3 amino acids to about 5 amino acids, for example the tetrapeptides: L-Ala-γ-D-Glu-Diaminopimelic acid-D-Ala (Tetrapeptide DAP), UDP-MurNAc-L-Ala-γ-D-Glu-Diaminopimelic acid-D-Ala (UDP-MurNAc-tetrapeptide) and MurNAc-L-Ala-γ-D-Glu-Diaminopimelic acid-D-Ala (MurNAc-tetrapeptide).

Templin et al. demonstrated that bacterially-derived UDP-MurNAc-tetrapeptide DAP, MurNAc-tetrapeptide DAP, and tetrapeptide DAP were all equally good substrates for E. coli LDC activity. Synthetic peptide substrates may be easier to use however, it should be understood that native substrate could be purified from bacteria and is also envisioned within the scope of this invention. The Lys-containing synthetic substrate is significantly less expensive than the diaminopimelic acid-containing synthetic substrate and was therefore the substrate of choice, but use of the diaminopimelic acid-containing synthetic substrate is also envisioned within the scope of this invention.

After the LDC enzyme cleaves the peptide substrate, it liberates D-Alanine which is then oxidized by a second enzyme, such as D-amino acid oxidase which produces hydrogen peroxide. In the presence of a third enzyme such as horse radish peroxidase, hydrogen peroxide reacts with a fluorogenic horseradish peroxidase substrate, for example Amplex Red, QuantaBlu, or o-phenylenediamine and produces a fluorescent product. Where the fluorogenic horseradish peroxidase substrate is Amplex Red, the reaction produces fluorescent resorufin. The reaction is incubated for a period of time at a given temperature, for example 30 minutes at 37 degrees Celsius. The incubation period can be varied from about 10 minutes to about 2 hours. As described supra, the incubation temperature can be varied independently from incubation period or pH and can range from about 35 degrees Celsius to about 40 degrees Celsius. Those skilled in the art will recognize that the incubation period and temperature can be modified without undue experimentation. The reaction is measured on a Microplate Spectrofluorometer such as the Molecular Devices SpectraMax Gemini with an excitation of wavelength of 530 nm (for Amplex Red) and an emission is detected at a wavelength of 590 nm (for Amplex Red). The temperature of the Spectrofluorometer can be adjusted to any temperature desired and can be the activation temperature of the LDC enzyme, such as 37 degrees Celcius, or a temperature above or below the activation temperature.

Therefore, another aspect of the invention is a method of identifying a compound that decreases the activity of an L-D Carboxypeptidase A enzyme, such as a PA LDC or the LDC of another bacterial species comprising:

(a) contacting the LDC enzyme in a buffer, for example 100 mM Tris at a pH 8.5, with a test compound, for example 2-Benzofurancarbothioic acid, S,S′-2,3-quinoxalinediyl ester, or 3,4-dihydro-g,3-dioxo-2H-1,4-Benzoxazine-6-crotonic acid,

(b) adding buffer solution, for example 100 mM Tris at pH 8.5 containing a suitable substrate, for example: L-Ala-γ-D-Glu-Diaminopimelic acid-D-Ala (Tetrapeptide DAP), UDP-MurNAc-L-Ala-γ-D-Glu-Diaminopimelic acid-D-Ala (UDP-MurNAc-tetrapeptide) and MurNAc-L-Ala-γ-D-Glu-Diaminopimelic acid-D-Ala (MurNAc-tetrapeptide).

(c) adding a second enzyme, such as D-amino acid oxidase to produce hydrogen peroxide

(d) adding a third enzyme, such as horse radish peroxidase and a fluorogenic horseradish peroxidase substrate (for example Amplex Red, QuantaBlu, or o-phenylenediamine) to react with the hydrogen peroxide; and

(e) measuring the resulting product (for example fluorescent resorufin) using a fluorometer.

After a compound has been identified that decreases PA LDC activity or expression, the compound can then be subjected to, directed or random chemical modifications such as acylation, alkylation, esterification, amidation, etc. to produce structural analogs of the agents. Candidate compounds can be selected randomly or can be based on existing compounds that bind to and/or inhibit the function of LDC activity. Therefore, a source of candidate agents is one or more library of molecules based on one or more known compounds that decreases LDC protein expression and/or enzyme activity in which the structure of the compound is changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions. One of ordinary skill in the art in the preparation of combinatorial libraries can readily prepare such libraries based on the existing compounds.

A variety of other reagents also can be included in a mixture with a compound in the assays of this invention. These include reagents such as salts, buffers, neutral proteins (e.g., albumin), detergents, etc. that can be used to facilitate optimal protein-protein and/or protein-nucleic acid binding. Such a reagent or reagents can also reduce non-specific or background interactions of the reaction components. Other reagents that improve the efficiency of the assay such as nuclease inhibitors, antimicrobial agents, and the like can also be used.

After a compound has been identified that decreases PA LDC activity or expression and/or after subsequent modifications, the compound can then be subjected administered to a bacterial cell, bacterial culture, or an organism infected with a cell expressing an LDC, such as PA LDC. This can be useful to establish toxicity and other pharmacological parameters of the compound important for determining dosing regimens. For example, after a compound is identified using an ex vivo system containing a PA LDC polypeptide, the compound can be administered to a culture of Pseudomonas aeruginosa or an organism infected with Pseudomonas aeruginosa to examine various pharmacological aspects of the compound. Exemplary compounds identified using the method herein described include:2-Benzofurancarbothioic acid, S,S′-2,3-quinoxalinediyl ester, and 3,4-dihydro-g,3-dioxo-2H-1,4-Benzoxazine-6-crotonic acid.

Therefore, in another embodiment, the invention provides a method of identifying a compound useful for causing bacterial lysis, comprising the steps of: (a) contacting a test compound with a PA LDC enzyme; and (b) determining whether the test compound decreases the activity of the PA LDC enzyme. In some embodiments, the method further comprises the steps of: (a) administering a therapeutically effective amount of a test compound to an animal with a bacterial infection; and (b) determining the extent to which the test compound inhibits bacterial growth or viability.

In some embodiments, the animal model of infection involves a rodent, for example, a rat or mouse; in another aspect the animal model of infection involves a dog, or a primate, for example, in clinical trials, a human.

Therapeutic efficacy and toxicity can be determined by standard pharmaceutical procedures in cell cultures or experimental animals by calculating, for example, the ED₅₀ (the dose therapeutically effective in 50% of the population) and the LD₅₀ (the dose lethal to 50% of the population). The dose ratio between toxic and therapeutic effects is the therapeutic index, and it can be expressed as the ratio, LD₅₀/ED₅₀. The data obtained from cell culture assays using PA LDC polypeptide, cells expressing PA LDC and/or animal studies, such as canine or primate studies, is used in formulating a range of dosage for human use. The dosage contained in such compositions preferably gives rise to a range of circulating concentrations that include the ED₅₀ with little or no toxicity. The dosage varies within this range depending upon the dosage form employed, sensitivity of the patient and the route of administration.

The exact dosage will be determined by the one administering the dose, in light of factors related to the subject requiring treatment. Dosage and administration are adjusted to provide sufficient levels of the active agent or to maintain the desired effect, for example, control of bacterial during stationary phase. Factors that can be taken into account include the severity of the infection and other factors, including the general health of the subject, age, weight and gender of the subject, diet, time and frequency of administration, drug combination(s), reaction sensitivities and tolerance/response to therapy.

The pharmaceutical compositions containing a compound that has been identified as inhibiting PA LDC activity can be administered by any number of routes including, but not limited to, oral, intravenous, intramuscular, intruarticular, intraarterial, intramedullary, intrathecal, epidural, intraventricular, transdermal, subcutaneous, intraperitoneal, intranasal, enteral, topical, sublingual, inhalational, intraocular, intra-aural or rectal means.

In addition to the active ingredients, these pharmaceutical compositions can contain suitable, pharmaceutically acceptable carriers comprising excipients and auxiliaries which facilitate processing of the active compounds into preparations that can be used pharmaceutically or which facilitate absorption or distribution of the active compounds. Further details on techniques for formulation and administration can be found in Remington's Pharmaceutical Sciences, Maack Publishing Co., Easton, Pa.

Pharmaceutical compositions for oral administration can be formulated using pharmaceutically acceptable carriers well-known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Compounds identified as either inhibiting PA LDC expression or PA LDC activity using the methods of the present invention, are also useful for the treatment of medical devices. PA LDC inhibitors kill bacteria, reduce viability and prevent the formation of biofilms. Since PA LDC inhibitors induce autolysis in P. aeruginosa, treating medical devices and implants such as catheters, sutures, contact lenses, orthopedic devices, mechanical heart valves, shunts, and grafts will help to reduce the chance of spreading infection. The LDC inhibitors can be applied directly to the surface of the device or implant by spraying, dipping, or the use of any coating technique known in the art. In another embodiment the PA LDC inhibitor can be incorporated directly into the medical device or implant itself. For example the PA LDC inhibitor can comprise one of the compounds in a mixture used to make a contact lens, shunt, bioimplant, graft, stent, heart valve or other implantable medical device.

### EXAMPLE 1

Cloning and Expression of PA LDC

Strain P. aeruginosa PA01 was streaked to single colonies on a trypticase soy agar plate and incubated at 37° C. overnight. As the source of template genomic DNA, ten colonies were scraped with a sterile inoculating loop into 50 μl of sterile water and boiled for 2 minutes. SEQ ID NO: 3 Primers PA_upl (5′CGCTACTAACATATGACCTCTCGTCCCTCCTCCGACC 3′) and SEQ ID NO: 4 PA_down1 (5′ CGCGGGATCCTACCAGCGCAACCGGTTTCCCTC 3′) were designed for Polymerase Chain Reaction (PCR) cloning of the LDC homolog from strain PA01 and were purchased from Integrated DNA Technologies, Inc. (Coralville, Iowa). The primers contain NdeI and BamHI restriction sites, respectively (as underlined); the ATG start and TAG stop codons are indicated (in bold). The primers were resuspended in water to 200 μM and further diluted to 10 μM. Reactions using the High Fidelity PCR Master kit (Roche Indianapolis, Ind.) consisted of 5 μl of each 10 μM primer, 2 μl of template DNA, 13 μl of water, and 25 μl of High Fidelity PCR Master. PCR was performed on the Applied Biosystems (Foster City, Calif.) GeneAmp PCR System 9700, using 5 minutes hold at 94° C., followed by 30 cycles of 1 minute 94° C., 1 minute 70° C., and 1 minute72° C., with a final 10 minutes at 72° C. The expected 0.9 kb PCR product was detected by agarose gel electrophoresis and was purified using the QIAquick PCR Purification Kit (QIAGEN, Inc., Valencia, Calif.), cleaved with restriction enzymes NdeI and BamHI, repurified with QIAquick, and ligated into the NdeI/BamHI sites of pET14b (Novagen, EMD Biosciences, Madison, Wis.). The vector pET14b places a hexahistidine tag on the amino terminus of a gene cloned into it. The vector contains a T7 promoter so that expression of the cloned gene can be induced by addition of isopropyl β-D-thio galactopyranoside (IPTG). The ligation mixture was transformed into E. coli using Novablue Singles Competent Cells (Novagen, EMD Biosciences, Madison, Wis.). Ampicillin resistant plasmid was prepared using the QIAGEN Plasmid Midi Kit and subjected to DNA sequence analysis (ACGT, Inc., Wheeling, Ill.).

The cloned DNA sequence (SEQ ID NO: 1) was translated and the amino acid sequence PA5198 (SEQ ID NO:2) was aligned with the sequence of a hypothetical protein of P. aeruginosa PA01, deposited in GenBank, as accession number AAG08583.1. The hypothetical protein was derived from the complete genome sequencing of P. aeruginosa. The hypothetical protein AAG08583.1 had not been isolated, expressed or characterized (Stover et al. Nature. Aug. 31, 2000; 406(6799):947-8).

As described supra, PA5198 (SEQ ID NO:2) shared only a 25% identity and a 35% similarity with the E coli LDC. Until now, comparing the function of the E. coli LDC and the P. aeruginosa AAG08583.1 putative protein, published in Genebank would not have been suggested or expected because of their low sequence homology, nor would it have been possible since the gene had not been cloned.

Plasmids containing the P. aeruginosa LDC insert were transformed into the E. coli expression strain, BL21. A 100 ml culture of BL21 harboring plasmid pPALDC1 was grown to mid-log (A₆₀₀ of 0.5), and PA LDC protein expression was induced by addition of Isopropyl-beta-D-thiogalactopyranoside (IPTG) to a final concentration of 0.4 mM as recommended (Novagen, EMD Biosciences, Madison, Wis.). After 3 hours of induction, cells were pelleted by centrifugation (10 minutes at 10 g), and the pellet was resuspended in Bugbuster containing Benzonase as recommended by the supplier (Novagen, EMD Biosciences, Madison, Wis.). The supernatant was applied to a Pharmacia HiPrep 26/10 desalting column (Amersham Biosciences, Piscataway, N.J.). The desalted sample in wash buffer (50 mM Tris/300 mM NaCl pH 7.5) was applied to a Talon column (Clontech, BD Biosciences, Palo Alto, Calif.), and eluted in wash buffer containing 150 mM imidazole. The purified PA LDC was dialyzed into 100 mMTris pH 8.5 containing 5% glycerol and was frozen in aliquots at −70° C. and used in LDC assays as described below.

The production of the PA LDC protein by the E. coli expression strain BL21 (Novagen, EMD Biosciences, Madison, Wis.) harboring the PA LDC plasmid was confirmed by visualization on a polyacrylamide gel. One hundred microliters of cells before and after induction with IPTG for 3 hours were loaded onto a polyacrylamide gel. A Coomassie-blue stain of a the polyacrylamide gel was performed and a prominent band of the predicted mass of 36 kDa was visible after induction with IPTG.

### EXAMPLE 2

LDC Assay

The assay consists of two parts: (i) cleavage of peptide substrate by LDC enzyme, and (ii) detection of the D-Ala cleavage product using Amplex Red. Conditions for E. coli LDC cleavage were established using a modification of conditions presented by Templin et al. (1999). The cleavage assay was assembled in black 96 half area flat bottom plates (Costar, 3694) (Corning Life Sciences, Acton, Mass.) and consisted of LDC at the indicated concentration in 30 μl (microliters) of 100 mM (milliMolar) Tris-HCl pH 8.5 containing 25 μg/ml of bovine serum albumin (B4287 Sigma, St. Louis, Mo.). “Tetrapeptide Lys” substrate L-Ala-γ-D-Glu-L-Lys-D-Ala was synthesized by SynPep, Inc. (Dublin, Calif.). Preliminary experiments (data not shown) demonstrated that this substrate (found in Gram positive bacteria and which is not the usual substrate for Gram negative bacteria such as E. coli and P. aeruginosa) is cleaved as efficiently as the Gram negative substrate L-Ala-γ-D-Glu-Diaminopimelic acid-D-Ala (Tetrapeptide DAP) used by Templin et al. The tetrapeptide Lys substrate (10 μl of 2 mM peptide in 100 mM Tris-HCl pH 8.5) was added to the enzyme, and the cleavage reaction was incubated for 30 minutes at 37° C. Inhibitor compounds were added to the enzyme in 2 μl of 30% dimethyl sulfoxide and incubated for 10 minutes at room temperature, prior to the addition of tetrapeptide. The negative control received 2 μl of 30% dimethyl sulfoxide alone without any compound.

In Templin et al., E. coli LDC activity was detected by reverse phase HPLC, a method which the present inventors considered to be unsuitable for large numbers of samples. The method of Gutheil et al. (2000) used a time consuming coupled assay to detect the D-Ala generated by the penicillin-binding protein and vancomycin activity assays. Furthermore, the Gutheil et al. (2000) method required a peptide purified from bacterial cultures, which is a laborious and low-yield procedure.

Alternatively, a detection method for the D-Ala product liberated by LDC cleavage of the tetrapeptide substrate was developed that would be easier to perform and the assay could comprise a synthetic substrate. A synthetic tetrapeptide substrate can easily be produced or purchased with any desired sequence or chemical modifications to the peptides from a company such as SynPep, Inc. (Dublin, Calif.).

The principle of the detection system is oxidation of D-Ala by the enzyme D-amino acid oxidase to produces hydrogen peroxide. In the presence of the enzyme horse radish peroxidase, hydrogen peroxide reacts with the fluorogenic horseradish peroxidase substrate Amplex Red, producing the fluorescent product, resorufin. The detection reaction which has now been optimized, consists of the addition of 40 μl (microliters) of detection reagent [12.5 units/ml horseradish peroxidase (P6140, Sigma St. Louis, Mo.), 0.38 units/ml D-amino acid oxidase (Sigma A5222), 6.25 μg/ml flavin adenine dinucleotide (F6625, Sigma St. Louis, Mo.), and 50 μM (micro Molar) Amplex Red (A-22177, Molecular Probes, Eugene, Oreg.) in 100 mM (miliMolar) Tris-HCl pH 8.5] to the completed cleavage reaction. The detection reaction was incubated for 30 minutes at 37° C., and the plates were read on a SpectraMax Gemini (excitation 530 nm, emission 590 nm) (Molecular Devices, Sunnyvale, Calif.).

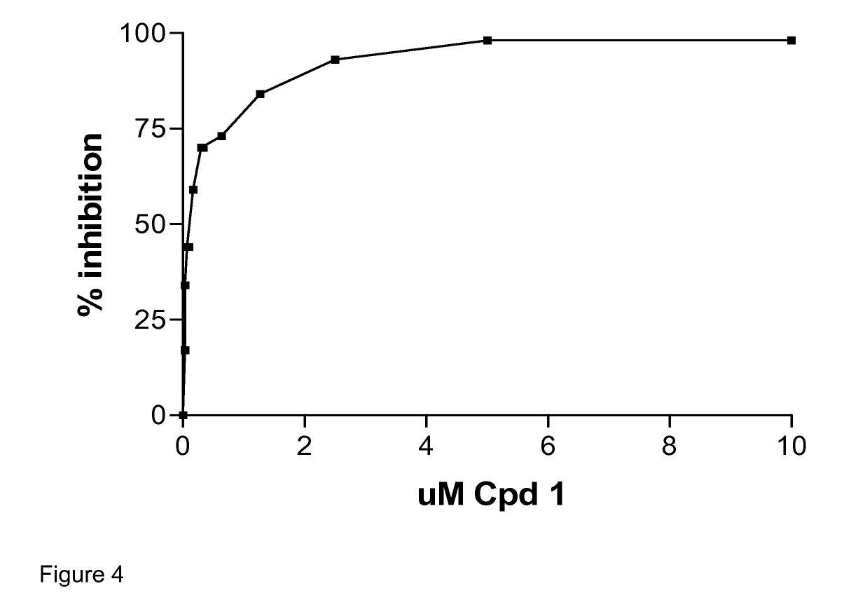

To demonstrate the feasibility of detecting inhibitors of PA LDC using the purified PA LDC enzyme and the aforementioned detection system, several inhibitors previously shown to be inhibitory to E. coli LDC were tested against PA LDC. The inhibition of PA LDC with the compound 2-Benzofurancarbothioic acid, S,S′-2,3-quinoxalinediyl ester, CAS Registry #253328-58-4 was shown with an IC₅₀ value of 0.1 μM. (E. coli IC₅₀=1.4 μM, FIG. 4). It was also shown that 3,4-dihydro-g,3-dioxo-2H-1,4-Benzoxazine-6-crotonic acid (CAS# 26518-87-6) inhibited PA LDC with an IC₅₀ of 170 μM (E. coli IC₅₀=12 μM, data not shown).

### EXAMPLE 3

To determine whether LDC inhibitors identified by the Amplex Red assay affect bacterial survival in the stationary phase, a microplate reader is used to monitor bacterial growth over time. An example of such a reader is the Bioscreen C (Growth Curves USA, Piscataway, N.J.). This instrument allows repeated reading of the absorbance of multiple cultures over time, with incubation and shaking at the desired temperature and speed, respectively.

In one embodiment of the experiment, a stationary culture of P. aeruginosa in LB is diluted 50 fold with fresh LB. One hundred μl of the diluted culture is applied to replicate wells of a Bioscreen plate. The LDC inhibitor being screened is added at the desired concentration(s) to wells. The Bioscreen is programmed to measure absorbance of these diluted cultures over time (e.g., every 30 minutes) to generate a “growth curve”. LDC inhibitors with the desired mechanism of action permits exponential growth to occur. Upon attainment of the stationary phase of growth, a drop in absorbance due to lysis of cells is expected to occur due to the inhibition of LDC by the compound being screened. Such lysis occurs in an E. coli strain deleted of the LDC gene (Templin et al.). After the attainment of the stationary phase of growth, a sample of the P. aeruginosa cells can be cultured to determine if recovery from the stationary phase has been affected or if the cells are otherwise viable. Additionally, this assay can be used to verify the activity of the LDC inhibitor being screened should a drop in absorbance not occur upon attainment of the stationary phase of growth.

1. A substantially purified polypeptide capable of LDC activity and having at least 96% sequence identity to SEQ ID NO:2.

2. A nucleic acid probe of at least 30 nucleotides but less than 924 nucleotides that selectively hybridizes to a polynucleotide encoded by SEQ ID NO:1 under stringent hybridization conditions.

3. A nucleic acid probe selected from: SEQ ID NO:3 and SEQ ID NO:4, that selectively hybridizes to a polynucleotide encoded by SEQ ID NO:1 under stringent hybridization conditions.

4. A polypeptide corresponding to 20 contiguous amino acids of SEQ ID NO:2.

5. An antibody that specifically binds to a polypeptide capable of LDC activity and wherein the polypeptide has at least 96% sequence identity to SEQ ID NO:2.

6. The antibody of claim 5, wherein the antibody is a polyclonal antibody or a monoclonal antibody.

7. A kit comprising an antibody of claim 6.

8. A method for detecting PA LDC polynucleotide in a sample, comprising the steps of:
(a) contacting the polynucleotide with a nucleic acid probe of at least 30 nucleotides but less than 924 nucleotides that selectively hybridizes to the polynucleotide under stringent hybridization conditions; and
(b) detecting the polynucleotide with the nucleic acid probe.

9. A method of detecting a polypeptide capable of LDC activity and having at least 96% sequence identity to SEQ ID NO: 2. comprising the steps of:
(a) contacting the polypeptide with an antibody that specifically binds to a polypeptide capable of LDC activity and having at least 96% sequence identity to SEQ ID NO: 2 and
(b) detecting the polypeptide with the antibody.

10. A method of identifying a compound that decreases the activity of an LDC enzyme, comprising the steps of:
(a) contacting the LDC enzyme with a test compound; and
(b) detecting the level of enzyme activity.

11. The method of claim 10 wherein the LDC enzyme comprises a polypeptide having an amino acid sequence of SEQ ID NO: 2.

12. The method of claim 11 wherein the contacting step further comprises the step of contacting the test compound with a cell expressing the LDC enzyme.

13. The method of claim 12 wherein the cell encodes a recombinant LDC enzyme.

14. The method of claim 12 wherein the cell endogenously expresses an LDC enzyme.

15. A method of identifying a compound that decreases the activity of an LDC enzyme, comprising the steps of:
(a) contacting the LDC enzyme with a test compound
(b) incubating the LDC enzyme in a buffer solution comprising a substrate;
(c) adding a second enzyme to produce hydrogen peroxide;
(d) adding a third enzyme and a fluorogenic horseradish peroxidase substrate to react with the hydrogen peroxide; and
(e) measuring a resulting product in a fluorometer

16. The method of claim 15, further comprising incubating the enzyme at a LDC enzyme-activating temperature from about 35° C. to about 39° C.

17. The method of claim 16, wherein the second enzyme is D-amino acid oxidase.

18. The method of claim 17, wherein the third enzyme is horse radish peroxidase.

19. The method of claim 18, wherein the fluorogenic horseradish peroxidase substrate is Amplex Red.

20. The method of claim 19, wherein the LDC enzyme is of Pseudomonas aeruginosa.

21. The method of claim 19, wherein the LDC enzyme is of E. coli.

22. A method of identifying a compound useful for causing bacterial lysis in the stationary phase of bacterial growth, comprising the steps of:
(a) contacting an LDC enzyme with a test compound;
(b) determining whether the test compound decreases the activity of the enzyme;
(c) administering the test compound to a bacterial culture; and
(d) measuring bacterial cell lysis in the culture.

23. A method of identifying a compound useful for inhibiting bacterial growth or viability, comprising the steps of:
(a) contacting a PA LDC enzyme with a test compound;
(b) determining whether the test compound decreases the activity of the enzyme;
(c) administering the test compound to an animal; and
(d) determining the extent to which the test compound reduces the bacterial infection of the animal.

24. A method of inducing bacterial lysis, comprising one or more of the steps selected from:
(a) decreasing the expression of a PA LDC gene; and
(b) contacting a PA LDC enzyme with a compound which decreases the activity of the enzyme.

25. A method for inhibiting bacterial growth or viability, comprising one or more of the steps selected from:
(a) decreasing the expression of a PA LDC gene; and
(b) contacting a PA LDC enzyme with a compound which decreases the activity of the enzyme.

26. A method for inhibiting bacterial growth or viability, comprising administering an effective dose of a PA LDC inhibitor selected from:
(a) 2-Benzofurancarbothioic acid, S,S′-2,3-quinoxalinediyl ester, and
(b) 3,4-dihydro-g,3-dioxo-2H-1,4-Benzoxazine-6-crotonic acid.

27. A method of inducing bacterial lysis, comprising administering an effective dose of a PA LDC inhibitor selected from:
(a) 2-Benzofurancarbothioic acid, S,S′-2,3-quinoxalinediyl ester, and (b) 3,4-dihydro-g,3-dioxo-2H-1,4-Benzoxazine-6-crotonic acid.

28. A method of treating a Pseudomonas aeruginosa infection in a patient, comprising administering an effective dose of a PA LDC inhibitor selected from:
(a) 2-Benzofurancarbothioic acid, S,S′-2,3-quinoxalinediyl ester, and
(b) 3,4-dihydro-g,3-dioxo-2H-1,4-Benzoxazine-6-crotonic acid.

29. A device comprising a therapeutically effective amount of an LDC inhibitor effective to inhibit bacterial growth wherein the device is selected from: a catheter, a stent, a suture, a contact lens, an orthopedic device, a mechanical heart valve, a shunt, and a graft.


SEQUENCE LISTING (7 sequences)

SEQ ID NO: 1 - DNA, 924 bp, Pseudomonas aeruginosa
atgacctctcgtccctcctccgaccagacttggcagcccatcgacggccgggtggcgctgatcgcgccagcctcggccatcgccaccgacgtgctggaggcgaccctgcggcaactggaagtgcacggcgtggactaccatctcggccgccacgtcgaagcccgctaccgctacctcgccgggacggtcgagcagcgcctggaagacctgcacaacgccttcgacatgccggacatcaccgcggtctggtgcctgcgcggcggctacggctgcggccagttgctgccgggcctggactgggggcgcctgcaggcggcctctccgcggccactgatcggtttctccgacatctcggtgttgctcagcgctttccaccgccacggcctgcctgcgatccacggcccggtggccaccggactcggtctgtcgccgctgagcgccccccgcgagcagcaggagcggctagcctctctggcctcggtcagccgcttgctggccggaatcgaccatgaactcccggtgcagcacctcggcggacacaagcaacgcgtcgaaggcgcgctgatcggcggcaacctcaccgccctggcctgcatggccggcacactcggcggcctgcacgccccggcggggtcgatcctggtcctggaggacgtcggcgaaccctactaccgcctggagcgcagcctctggcaattgctcgaaagcatcgacgcgcgccagctcggcgcgatctgcctgggctcgttcaccgattgcccgcgcaaggaagtcgcccacagcctggagcggatcttcggcgaatacgccgccgccatcgaggtgccgctctaccaccacctgcccagcgggcatggtgcgcagaaccgagcctggccttacgggaagaccgcggtgctcgagggaaaccggttgcgctggtag

SEQ ID NO: 2 - protein, 307 aa, Pseudomonas aeruginosa
MTSRPSSDQTWQPIDGRVALIAPASAIATDVLEATLRQLEVHGVDYHLGRHVEARYRYLAGTVEQRLEDLHNAFDMPDITAVWCLRGGYGCGQLLPGLDWGRLQAASPRPLIGFSDISVLLSAFHRHGLPAIHGPVATGLGLSPLSAPREQQERLASLASVSRLLAGIDHELPVQHLGGHKQRVEGALIGGNLTALACMAGTLGGLHAPAGSILVLEDVGEPYYRLERSLWQLLESIDARQLGAICLGSFTDCPRKEVAHSLERIFGEYAAAIEVPLYHHLPSGHGAQNRAWPYGKTAVLEGNRLRW

SEQ ID NO: 3 - DNA, 37 bp, Artificial Sequence
  Description of Artificial Sequence Synthetic primer
cgctactaacatatgacctctcgtccctcctccgacc

SEQ ID NO: 4 - DNA, 33 bp, Artificial Sequence
  Description of Artificial Sequence Synthetic primer
cgcgggatcctaccagcgcaaccggtttccctc

SEQ ID NO: 5 - protein, 304 aa, Escherichia coli [sequence omitted]

SEQ ID NO: 6 - DNA, 1000 bp, Pseudomonas aeruginosa
  CDS (10)..(990)
atattactcatgggcagcagccatcatcatcatcatcacagcagcggcctggtgccgcgcggcagccatatgacctctcgtccctcctccgaccagacttggcagcccatcgacggccgggtggcgctgatcgcgccagcctcggccatcgccaccgacgtgctggaggcgaccctgcggcaactggaagtgcacggcgtggactaccatctcggccgccacgtcgaagcccgctaccgctacctcgccgggacggtcgagcagcgcctggaagacctgcacaacgccttcgacatgccggacatcaccgcggtctggtgcctgcgcggcggctacggctgcggccagttgctgccgggcctggactgggggcgcctgcaggcggcctctccgcggccactgatcggtttctccgacatctcggtgttgctcagcgctttccaccgccacggcctgcctgcgatccacggcccggtggccaccggactcggtctgtcgccgctgagcgccccccgcgagcagcaggagcggctagcctctctggcctcggtcagccgcttgctggccggaatcgaccatgaactcccggtgcagcacctcggcggacacaagcaacgcgtcgaaggcgcgctgatcggcggcaacctcaccgccctggcctgcatggccggcacactcggcggcctgcacgccccggcggggtcgatcctggtcctggaggacgtcggcgaaccctactaccgcctggagcgcagcctctggcaattgctcgaaagcatcgacgcgcgccagctcggcgcgatctgcctgggctcgttcaccgattgcccgcgcaaggaagtcgcccacagcctggagcggatcttcggcgaatacgccgccgccatcgaggtgccgctctaccaccacctgcccagcgggcatggtgcgcagaaccgagcctggccttacgggaagaccgcggtgctcgagggaaaccggttgcgctggtaggatccgg

SEQ ID NO: 7 - protein, 327 aa, Pseudomonas aeruginosa
MGSSHHHHHHSSGLVPRGSHMTSRPSSDQTWQPIDGRVALIAPASAIATDVLEATLRQLEVHGVDYHLGRHVEARYRYLAGTVEQRLEDLHNAFDMPDITAVWCLRGGYGCGQLLPGLDWGRLQAASPRPLIGFSDISVLLSAFHRHGLPAIHGPVATGLGLSPLSAPREQQERLASLASVSRLLAGIDHELPVQHLGGHKQRVEGALIGGNLTALACMAGTLGGLHAPAGSILVLEDVGEPYYRLERSLWQLLESIDARQLGAICLGSFTDCPRKEVAHSLERIFGEYAAAIEVPLYHHLPSGHGAQNRAWPYGKTAVLEGNRLRW